(19) United States
(12) Patent Application Publication
(10) Pub. No.: US 2025/0244325 A1
(43) Pub. Date: Jul. 31, 2025

(54) ANTI-SARS-COV-2 ANTIBODIES AND USES THEREOF II

(71) Applicant: Seqirus Pty Ltd, Melbourne (AU)

(72) Inventors: Steven ROCKMAN, Victoria (AU); Kirsten VANDENBERG, Victoria (AU); Jesse BODLE, Victoria (AU)

(21) Appl. No.: 18/698,870

(22) PCT Filed: Oct. 7, 2022
(86) PCT No.: PCT/AU2022/051203

§ 371 Date: Apr. 5, 2024

(30) Foreign Application Priority Data
Oct. 7, 2021 (AU) 2021903206

(51) Int. Cl. G01N 33/569; C07K 16/10; C12Q 1/28; G01N 33/543; G01N 33/68

(52) U.S. Cl. CPC G01N 33/56983; C07K 16/1003; C12Q 1/28; G01N 33/543; G01N 33/6893; C07K 2317/565; G01N 2333/165; G01N 2333/908; G01N 2469/10

(57) ABSTRACT

The present disclosure relates to anti-SARS-COV-2 antibodies and uses thereof in detecting intact multimeric and/or intact trimeric severe acute respiratory syndrome coronavirus 2 (SARS-COV-2) spike protein in a sample.

### RELATED APPLICATION DATA

The present application claims priority from Australian Patent Application No. 2021903206 filed 7 Oct. 2021 entitled “Anti-SARS-COV-2 Antibodies and Uses Thereof II”, the entire contents of which is hereby incorporated by reference.

### SEQUENCE LISTING

The present application is filed together with a Sequence Listing in electronic form. The entire contents of the Sequence Listing are hereby incorporated by reference.

### FIELD

The present disclosure relates to anti-SARS-COV-2 antibodies and uses thereof in detecting intact multimeric and/or intact trimeric severe acute respiratory syndrome coronavirus 2 (SARS-COV-2) spike protein in a sample.

### BACKGROUND

In late 2019, a novel severe acute respiratory syndrome (SARS) virus was identified in China in humans. Since December 2019, the global spread of this virus has been widespread to all countries with the World Health Organisation declaring the outbreak a pandemic on Mar. 11, 2020. Human infection with this virus displays a broad clinical spectrum with a high rate of transmissibility. The global count of infections and mortality continues to rise with infections exceeding 615 million and over 6.54 million deaths to date.

Vaccines are the critical health intervention to prevent this infectious disease. This pandemic has seen the unprecedented development of multiple vaccines, with 8 vaccines approved for full use and over 130 in Phase 1-3 clinical trials. The majority of the vaccines that have been developed attempt to evoke the immune system to recognise the SARS-COV-2 spike protein (or S protein) since early studies of recombinant SARS-CoV protein in a hamster challenge model demonstrated that this approach was immunogenic and protective.

However, the development of effective vaccines to counter the spread of SARS-Cov-2 (and treat the associated severely infectious coronavirus disease 2019 (COVID-19)) relies on the ability to discriminate between the level of denatured or monomeric protein, which is considered a poor immunogen, from intact protein. To date, no such assay exists. Thus, it will be apparent to the skilled person that there remains a need for a test with adequate sensitivity and specificity to detect an immunogenic composition comprising the S protein and in particular discriminate between intact protein and denatured or monomeric protein.

### SUMMARY

The present disclosure is based on the inventors' production of proteins comprising antibody variable regions that bind to intact multimeric and/or trimeric SARS-COV-2 spike(S) protein. In addition, the inventors have developed an ELISA-based capture-detect assay for the detection of intact multimeric and/or trimeric SARS-CoV-2 S protein using the proteins of the disclosure. Importantly, the inventors found that the proteins of the disclosure do not detect denatured or monomeric S protein, which is considered a poor immunogen. Thus, based on these findings the inventors have also developed a method to determine the potency of an immunogenic composition comprising a S protein.

The findings by the inventors provide the basis for a method for detecting intact multimeric and/or intact trimeric SARS-COV-2 S protein in a sample. The findings by the inventors also provide the basis for methods of determining the potency of an immunogenic composition comprising an S protein. Additionally, the findings by the inventors provide the basis for proteins comprising antibody variable regions that bind to intact multimeric and/or intact trimeric SARS-COV-2 S protein.

Accordingly, the present disclosure provides a method for detecting intact multimeric and/or intact trimeric SARS-COV-2 S protein in a sample, the method comprising:
(i) contacting a sample to a capture protein comprising an antibody variable region immobilized on a solid surface, wherein the immobilized capture protein binds to a first receptor binding domain (RBD) on a first S protein monomer unit of the multimeric and/or trimeric SARS-COV-2 S protein to thereby form a protein complex;
(ii) contacting the protein complex with a detection protein comprising an antibody variable region, wherein the detection protein binds to a second RBD on a second S protein monomer unit of the captured multimeric and/or trimeric SARS-COV-2 S protein and wherein the detection protein comprises a detectable label; and
(iii) detecting the label, wherein the presence of the label is indicative of the presence (iii) of intact multimeric and/or intact trimeric SARS-COV-2 S protein in the sample.

Accordingly, the present disclosure provides a method for detecting intact multimeric and/or intact trimeric SARS-COV-2 S protein in a sample, the method comprising:
(i) immobilizing a capture protein comprising an antibody variable region onto a solid surface;
(ii) contacting the sample to the capture protein comprising an antibody variable region immobilized on the solid surface, wherein the immobilized capture protein binds to a first receptor binding domain (RBD) on a first S protein monomer unit of the multimeric and/or trimeric SARS-COV-2 S protein to thereby form a protein complex;
(iii) contacting the protein complex with a detection protein comprising an antibody variable region, wherein the detection protein binds to a second RBD on a second S protein monomer unit of the captured multimeric and/or trimeric SARS-COV-2 S protein and wherein the detection protein comprises a detectable label; and
(iv) detecting the label, wherein the presence of the label is indicative of the presence of intact multimeric and/or intact trimeric SARS-COV-2 S protein in the sample.

The present disclosure also provides a method for detecting intact multimeric and/or intact trimeric SARS-COV-2 S protein in a sample, the method comprising:
(i) immobilizing a capture protein comprising an antibody variable region onto a solid surface;
(ii) contacting the sample to the capture protein comprising an antibody variable region immobilized on the solid surface, wherein the immobilized capture protein binds to a first RBD on a first S protein monomer unit of the multimeric and/or trimeric SARS-COV-2 S protein to thereby form a protein complex;
(iii) contacting the protein complex with a detection protein comprising an antibody variable region, wherein the detection protein binds to a second RBD on a second S protein monomer unit of the captured multimeric and/or trimeric SARS-COV-2 S protein;
(iv) contacting the detection protein with an antibody that binds thereto and comprises a detectable label; and
(v) detecting the label, wherein the presence of the label is indicative of the presence of intact multimeric and/or intact trimeric SARS-COV-2 S protein in the sample.

The present disclosure also provides a method for detecting intact multimeric and/or intact trimeric SARS-COV-2 S protein in a sample, the method comprising:
(i) contacting the sample to a capture protein comprising an antibody variable region immobilized on the solid surface, wherein the immobilized capture protein binds to a first RBD on a first S protein monomer unit of the multimeric and/or trimeric SARS-COV-2 S protein to thereby form a protein complex;
(ii) contacting the protein complex with a detection protein comprising an antibody variable region, wherein the detection protein binds to a second RBD on a second S protein monomer unit of the captured multimeric and/or trimeric SARS-COV-2 S protein;
(iii) contacting the detection protein with an antibody that binds thereto and comprises a detectable label; and
(iv) detecting the label, wherein the presence of the label is indicative of the presence of intact multimeric and/or intact trimeric SARS-COV-2 S protein in the sample.

In one example, the method additionally comprises providing the sample.

In one example, the method additionally provides after step (ii) the solid surface is washed to remove unbound protein. For example, after contacting the sample to the capture protein comprising an antibody variable region immobilized on the solid surface, the solid surface is washed to remove unbound protein.

In one example, the method additionally provides after step (iii) the solid surface is washed to remove unbound protein. For example, after contacting the protein complex with a detection protein comprising an antibody variable region, the solid surface is washed to remove unbound protein.

In one example, the sample is pre-treated with a zwittergent, a non-zwittergent or a non-detergent sulfobetaine. For example, the sample is pre-treated with a zwittergent. In one example, the zwittergent is a sulfonated zwittergent. In another example, the zwittergent is a non-sulfonated zwittergent. In one example, the sample is pre-treated with a non-zwittergent. In a further example, the sample is pre-treated with a non-detergent sulfobetaine.

In one example, the sample is pre-treated with a zwittergent for 3 hours at 37° C. The inventors of the present disclosure found that pre-treatment of the S protein containing sample at 37° C. for 3 hours normalizes the trimer ratio in the sample. Furthermore, the inventors of the disclosure found that pre-treatment of the sample with zwittergent reduces and/or disrupts the formation of aggregates to trimers. For example, the sample is pre-treated with at least about 0.025% zwittergent prior to adding to the solid surface. In one example, the sample is pre-treated with at least about 0.025%, or about 0.030%, or about 0.035%, or about 0.040%, or about 0.045%, or about 0.050%, or about 0.055%, or about 0.060%, or about 0.065%, or about 0.070% zwittergent for 3 hours at 37° C. For example, the sample is pre-treated with about 0.030-0.035% zwittergent for 3 hours at 37° C. For example, the sample is pre-treated with about 0.065% zwittergent for 3 hours at 37° C. In another example, the sample is pre-treated with at least about 0.100%, or about 0.105%, or about 0.110%, or about 0.115%, or about 0.120%, or about 0.125%, or about 0.130% zwittergent for 3 hours at 37° C. For example, the sample is pre-treated with about 0.125% zwittergent for 3 hours at 37° C. In a further example, the sample is pre-treated with at least about 0.15%, or about 0.20%, or about 0.25%, or about 0.30% zwittergent for 3 hours at 37° C. For example, the sample is pre-treated with about 0.25% zwittergent for 3 hours at 37° C.

In one example, the sample is pre-treated with a non-zwittergent for 3 hours at 37° C. For example, the sample is pre-treated with at least about 0.025% non-zwittergent prior to adding to the solid surface. In one example, the sample is pre-treated with at least about 0.025%, or about 0.030%, or about 0.035%, or about 0.040%, or about 0.045%, or about 0.050%, or about 0.055%, or about 0.060%, or about 0.065%, or about 0.070% non-zwittergent for 3 hours at 37° C. For example, the sample is pre-treated with about 0.030-0.035% non-zwittergent for 3 hours at 37° C. For example, the sample is pre-treated with about 0.065% non-zwittergent for 3 hours at 37° C. In another example, the sample is pre-treated with at least about 0.100%, or about 0.105%, or about 0.110%, or about 0.115%, or about 0.120%, or about 0.125%, or about 0.130% non-zwittergent for 3 hours at 37° C. For example, the sample is pre-treated with about 0.125% non-zwittergent for 3 hours at 37° C. In a further example, the sample is pre-treated with at least about 0.15%, or about 0.20%, or about 0.25%, or about 0.30% non-zwittergent for 3 hours at 37° C. For example, the sample is pre-treated with about 0.25% non-zwittergent for 3 hours at 37° C.

In one example, the sample is pre-treated with a non-detergent sulfobetaine for 3 hours at 37° C. For example, the sample is pre-treated with at least about 0.025% non-detergent sulfobetaine prior to adding to the solid surface. In one example, the sample is pre-treated with at least about 0.025%, or about 0.030%, or about 0.035%, or about 0.040%, or about 0.045%, or about 0.050%, or about 0.055%, or about 0.060%, or about 0.065%, or about 0.070% non-detergent sulfobetaine for 3 hours at 37° C. For example, the sample is pre-treated with about 0.030-0.035% non-detergent sulfobetaine for 3 hours at 37° C. For example, the sample is pre-treated with about 0.065% non-detergent sulfobetaine for 3 hours at 37° C. In another example, the sample is pre-treated with at least about 0.100%, or about 0.105%, or about 0.110%, or about 0.115%, or about 0.120%, or about 0.125%, or about 0.130% non-detergent sulfobetaine for 3 hours at 37° C. For example, the sample is pre-treated with about 0.125% non-detergent sulfobetaine for 3 hours at 37° C. In a further example, the sample is pre-treated with at least about 0.15%, or about 0.20%, or about 0.25%, or about 0.30% non-detergent sulfobetaine for 3 hours at 37° C. For example, the sample is pre-treated with about 0.25% non-detergent sulfobetaine for 3 hours at 37° C.

The present inventors also found that adding the sample to the solid surface in the presence of a zwittergent reduced high molecular weight (HMW) aggregates to trimers. In one example, the sample is added to the solid surface in the presence of zwittergent. For example, the sample is added to the solid surface in a diluent comprising a zwittergent. In one example, the sample is added to the solid surface in a diluent comprising at least about 0.100%, or about 0.105%, or about 0.110%, or about 0.115%, or about 0.120%, or about 0.125%, or about 0.130% zwittergent. For example, the sample is added to the solid surface in a diluent comprising 0.125% zwittergent.

In one example, the capture protein and/or the detection protein comprise a fragment variable (Fv). For example, the capture protein and/or the detection protein is selected from the group consisting of:
(i) a single chain Fv fragment (scFv);
(ii) a dimeric scFv (di-scFv);
(iii) a diabody;
(iv) a triabody;
(v) a tetrabody;
(vi) a fragment antigen binding (Fab);
(vii) a F(ab′)₂;
(viii) a Fv;
(ix) one of (i) to (viii) linked to a constant region of an antibody, a constant fragment (Fc) or a heavy chain constant domain (C_{H}) 2 and/or C_{H}3; and
(x) an antibody.

In one example, the capture protein comprises a Fv. For example, the capture protein is selected from the group consisting of:
(i) a scFv;
(ii) a di-scFv;
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, a Fc or a C_{H}2 and/or C_{H}3; and
(x) an antibody.

In one example, the detection protein comprises a Fv. For example, the detection protein is selected from the group consisting of:
(i) a scFv;
(ii) a di-scFv;
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, a Fc or a C_{H}2 and/or C_{H}3; and
(x) an antibody.

In one example, the capture protein and/or the detection protein is an antibody. For example, the capture protein is an antibody. In another example, the detection protein is an antibody. Exemplary antibodies are full-length and/or naked antibodies.

In one example, the capture protein and/or the detection protein is recombinant, chimeric, CDR grafted, humanized, synhumanized, primatized, deimmunized or human.

In one example, the capture protein and/or the detection protein is an antibody comprising:
(i) a heavy chain variable region (V_{H}) comprising:
   (a) a complementarity determining region (CDR) 1 comprising a sequence set forth in:
      a. amino acids 26 to 33 of SEQ ID NO: 1; or
      b. amino acids 26 to 33 of SEQ ID NO: 3; or
      c. amino acids 26 to 34 of SEQ ID NO: 5; or
      d. amino acids 26 to 33 of SEQ ID NO: 7; or
      e. amino acids 26 to 33 of SEQ ID NO: 49; or
      f. amino acids 26 to 33 of SEQ ID NO: 52; and
   (b) a CDR2 comprising a sequence set forth in:
      a. amino acids 51 to 58 of SEQ ID NO: 1; or
      b. amino acids 51 to 58 of SEQ ID NO: 3; or
      c. amino acids 52 to 58 of SEQ ID NO: 5; or
      d. amino acids 51 to 58 of SEQ ID NO: 7; or
      e. amino acids 51 to 58 of SEQ ID NO: 49; or
      f. amino acids 51 to 58 of SEQ ID NO: 52; and
   (c) a CDR3 comprising a sequence set forth in:
      a. amino acids 97 to 110 of SEQ ID NO: 1; or
      b. amino acids 97 to 110 of SEQ ID NO: 3; or
      c. amino acids 97 to 108 of SEQ ID NO: 5; or
      d. amino acids 97 to 106 of SEQ ID NO: 7; or
      e. amino acids 97 to 110 of SEQ ID NO: 49;
      f. amino acids 97 to 110 of SEQ ID NO: 52; and
(ii) a light chain variable region (V_{L}) comprising:
   (a) a CDR1 comprising a sequence set forth in:
      a. amino acids 27 to 32 of SEQ ID NO: 2; or
      b. amino acids 27 to 31 of SEQ ID NO: 4; or
      c. amino acids 27 to 38 of SEQ ID NO: 6; or
      d. amino acids 27 to 37 of SEQ ID NO: 8; or
      e. amino acids 27 to 36 of SEQ ID NO: 53; and
   (b) a CDR2 comprising a sequence set forth in:
      a. amino acids 50 to 52 of SEQ ID NO: 2; or
      b. amino acids 49 to 51 of SEQ ID NO: 4; or
      c. amino acids 56 to 58 of SEQ ID NO: 6; or
      d. amino acids 55 to 57 of SEQ ID NO: 8; or
      e. amino acids 54 to 56 of SEQ ID NO: 53; and
   (c) a CDR3 comprising a sequence set forth in:
      a. amino acids 89 to 97 of SEQ ID NO: 2; or
      b. amino acids 88 to 96 of SEQ ID NO: 4; or
      C amino acids 95 to 103 of SEQ ID NO: 6; or
      d. amino acids 94 to 102 of SEQ ID NO: 8; or
      e. amino acids 93 to 101 of SEQ ID NO: 53.

In one example, the capture protein is an antibody comprising:
(i) a heavy chain variable region (V_{H}) comprising:
   (a) a complementarity determining region (CDR) 1 comprising a sequence set forth in:
      a. amino acids 26 to 33 of SEQ ID NO: 1; or
      b. amino acids 26 to 33 of SEQ ID NO: 3; or
      c. amino acids 26 to 34 of SEQ ID NO: 5; or
      d. amino acids 26 to 33 of SEQ ID NO: 7; or
      e. amino acids 26 to 33 of SEQ ID NO: 49; or
      f. amino acids 26 to 33 of SEQ ID NO: 52; and
   (b) a CDR2 comprising a sequence set forth in:
      a. amino acids 51 to 58 of SEQ ID NO: 1; or
      b. amino acids 51 to 58 of SEQ ID NO: 3; or
      c. amino acids 52 to 58 of SEQ ID NO: 5; or
      d. amino acids 51 to 58 of SEQ ID NO: 7; or
      e. amino acids 51 to 58 of SEQ ID NO: 49; or
      f. amino acids 51 to 58 of SEQ ID NO: 52; and
   (c) a CDR3 comprising a sequence set forth in:
      a. amino acids 97 to 110 of SEQ ID NO: 1; or
      b. amino acids 97 to 110 of SEQ ID NO: 3; or
      c. amino acids 97 to 108 of SEQ ID NO: 5; or
      d. amino acids 97 to 106 of SEQ ID NO: 7; or
      e. amino acids 97 to 110 of SEQ ID NO: 49;
      f. amino acids 97 to 110 of SEQ ID NO: 52; and
(ii) a light chain variable region (V_{L}) comprising:
   (a) a CDR1 comprising a sequence set forth in:
      a. amino acids 27 to 32 of SEQ ID NO: 2; or
      b. amino acids 27 to 31 of SEQ ID NO: 4; or
      c. amino acids 27 to 38 of SEQ ID NO: 6; or
      d. amino acids 27 to 37 of SEQ ID NO: 8; or
      e. amino acids 27 to 36 of SEQ ID NO: 53; and
   (b) a CDR2 comprising a sequence set forth in:
      a. amino acids 50 to 52 of SEQ ID NO: 2; or
      b. amino acids 49 to 51 of SEQ ID NO: 4; or
      c. amino acids 56 to 58 of SEQ ID NO: 6; or
      d. amino acids 55 to 57 of SEQ ID NO: 8; or
      e. amino acids 54 to 56 of SEQ ID NO: 53; and
   (c) a CDR3 comprising a sequence set forth in:
      a. amino acids 89 to 97 of SEQ ID NO: 2; or
      b. amino acids 88 to 96 of SEQ ID NO: 4; or
      c. amino acids 95 to 103 of SEQ ID NO: 6; or
      d. amino acids 94 to 102 of SEQ ID NO: 8; or
      e. amino acids 93 to 101 of SEQ ID NO: 53.

In one example, the detection protein is an antibody comprising:
(i) a heavy chain variable region (V_{H}) comprising:
   (a) a complementarity determining region (CDR) 1 comprising a sequence set forth in:
      a. amino acids 26 to 33 of SEQ ID NO: 1; or
      b. amino acids 26 to 33 of SEQ ID NO: 3; or
      c. amino acids 26 to 34 of SEQ ID NO: 5; or
      d. amino acids 26 to 33 of SEQ ID NO: 7; or
      e. amino acids 26 to 33 of SEQ ID NO: 49; or
      f. amino acids 26 to 33 of SEQ ID NO: 52; and
   (b) a CDR2 comprising a sequence set forth in:
      a. amino acids 51 to 58 of SEQ ID NO: 1; or
      b. amino acids 51 to 58 of SEQ ID NO: 3; or
      c. amino acids 52 to 58 of SEQ ID NO: 5; or
      d. amino acids 51 to 58 of SEQ ID NO: 7; or
      e. amino acids 51 to 58 of SEQ ID NO: 49; or
      f. amino acids 51 to 58 of SEQ ID NO: 52; and
   (c) a CDR3 comprising a sequence set forth in:
      a. amino acids 97 to 110 of SEQ ID NO: 1; or
      b. amino acids 97 to 110 of SEQ ID NO: 3; or
      c. amino acids 97 to 108 of SEQ ID NO: 5; or
      d. amino acids 97 to 106 of SEQ ID NO: 7; or
      e. amino acids 97 to 110 of SEQ ID NO: 49;
      f. amino acids 97 to 110 of SEQ ID NO: 52; and
(ii) a light chain variable region (V_{L}) comprising:
   (a) a CDR1 comprising a sequence set forth in:
      a. amino acids 27 to 32 of SEQ ID NO: 2; or
      b. amino acids 27 to 31 of SEQ ID NO: 4; or
      c. amino acids 27 to 38 of SEQ ID NO: 6; or
      d. amino acids 27 to 37 of SEQ ID NO: 8; or
      e. amino acids 27 to 36 of SEQ ID NO: 53; and
   (b) a CDR2 comprising a sequence set forth in:
      a. amino acids 50 to 52 of SEQ ID NO: 2; or
      b. amino acids 49 to 51 of SEQ ID NO: 4; or
      c. amino acids 56 to 58 of SEQ ID NO: 6; or
      d. amino acids 55 to 57 of SEQ ID NO: 8; or
      e. amino acids 54 to 56 of SEQ ID NO: 53; and
   (c) a CDR3 comprising a sequence set forth in:
      a. amino acids 89 to 97 of SEQ ID NO: 2; or
      b. amino acids 88 to 96 of SEQ ID NO: 4; or
      c. amino acids 95 to 103 of SEQ ID NO: 6; or
      d. amino acids 94 to 102 of SEQ ID NO: 8; or
      e. amino acids 93 to 101 of SEQ ID NO: 53.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 1 or a CDR1 comprising a sequence set forth in amino acids 26 to 33 of SEQ ID NO: 49; and
   (b) a CDR2 comprising a sequence set forth in amino acids 51 to 58 of SEQ ID NO: 1 or a CDR2 comprising a sequence set forth in amino acids 51 to 58 of SEQ ID NO: 49; and
   (c) a CDR3 comprising a sequence set forth in amino acids 97 to 110 of SEQ ID NO: 1 or a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 1 or a CDR1 comprising a sequence set forth in amino acids 26 to 33 of SEQ ID NO: 49; and
   (b) a CDR2 comprising a sequence set forth in amino acids 51 to 58 of SEQ ID NO: 1 or a CDR2 comprising a sequence set forth in amino acids 51 to 58 of SEQ ID NO: 49; and
   (c) a CDR3 comprising a sequence set forth in amino acids 97 to 110 of SEQ ID NO: 1 or a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 1 or a CDR1 comprising a sequence set forth in amino acids 26 to 33 of SEQ ID NO: 49; and
   (b) a CDR2 comprising a sequence set forth in amino acids 51 to 58 of SEQ ID NO: 1 or a CDR2 comprising a sequence set forth in amino acids 51 to 58 of SEQ ID NO: 49; and
   (c) a CDR3 comprising a sequence set forth in amino acids 97 to 110 of SEQ ID NO: 1 or a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 1; and
   (b) a CDR2 comprising a sequence set forth in amino acids 51 to 58 of SEQ ID NO: 1; and
   (c) a CDR3 comprising a sequence set forth in amino acids 97 to 110 of SEQ ID NO: 1; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 1; and
   (b) a CDR2 comprising a sequence set forth in amino acids 51 to 58 of SEQ ID NO: 1; and
   (c) a CDR3 comprising a sequence set forth in amino acids 97 to 110 of SEQ ID NO: 1; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 1; and
   (b) a CDR2 comprising a sequence set forth in amino acids 51 to 58 of SEQ ID NO: 1; and
   (c) a CDR3 comprising a sequence set forth in amino acids 97 to 110 of SEQ ID NO: 1; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 49; and
   (b) a CDR2 comprising a sequence set forth in amino acids 51 to 58 of SEQ ID NO: 49; and
   (c) a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 49; and
   (b) a CDR2 comprising a sequence set forth in amino acids 51 to 58 of SEQ ID NO: 49; and
   (c) a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 49; and
   (b) a CDR2 comprising a sequence set forth in amino acids 51 to 58 of SEQ ID NO: 49; and
   (c) a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 32 of SEQ ID NO: 2; and
   (b) a CDR2 comprising a sequence set forth in amino acids 50 to 52 of SEQ ID NO: 2; and
   (c) a CDR3 comprising a sequence set forth in amino acids 89 to 97 of SEQ ID NO: 2.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3 or a CDR1 comprising a sequence set forth in amino acids 26 to 33 of SEQ ID NO: 49; and
   (b) a CDR2 comprising a sequence set forth in amino acids 51 to 58 of SEQ ID NO: 3 or a CDR2 comprising a sequence set forth in amino acids 51 to 58 of SEQ ID NO: 49; and
   (c) a CDR3 comprising a sequence set forth in amino acids 97 to 110 of SEQ ID NO: 3 or a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3 or a CDR1 comprising a sequence set forth in amino acids 26 to 33 of SEQ ID NO: 49; and
   (b) a CDR2 comprising a sequence set forth in amino acids 51 to 58 of SEQ ID NO: 3 or a CDR2 comprising a sequence set forth in amino acids 51 to 58 of SEQ ID NO: 49; and
   (c) a CDR3 comprising a sequence set forth in amino acids 97 to 110 of SEQ ID NO: 3 or a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3 or a CDR1 comprising a sequence set forth in amino acids 26 to 33 of SEQ ID NO: 49; and
   (b) a CDR2 comprising a sequence set forth in amino acids 51 to 58 of SEQ ID NO: 3 or a CDR2 comprising a sequence set forth in amino acids 51 to 58 of SEQ ID NO: 49; and
   (c) a CDR3 comprising a sequence set forth in amino acids 97 to 110 of SEQ ID NO: 3 or a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3; and
   (b) a CDR2 comprising a sequence set forth in amino acids 51 to 58 of SEQ ID NO: 3; and
   (c) a CDR3 comprising a sequence set forth in amino acids 97 to 110 of SEQ ID NO: 3; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3; and
   (b) a CDR2 comprising a sequence set forth in amino acids 51 to 58 of SEQ ID NO: 3; and
   (c) a CDR3 comprising a sequence set forth in amino acids 97 to 110 of SEQ ID NO: 3; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3; and
   (b) a CDR2 comprising a sequence set forth in amino acids 51 to 58 of SEQ ID NO: 3; and
   (c) a CDR3 comprising a sequence set forth in amino acids 97 to 110 of SEQ ID NO: 3; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 49; and
   (b) a CDR2 comprising a sequence set forth in amino acids 51 to 58 of SEQ ID NO: 49; and
   (c) a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 49; and
   (b) a CDR2 comprising a sequence set forth in amino acids 51 to 58 of SEQ ID NO: 49; and
   (c) a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 49; and
   (b) a CDR2 comprising a sequence set forth in amino acids 51 to 58 of SEQ ID NO: 49; and
   (c) a CDR3 comprising a sequence set forth in amino acids 97 to 110 of SEQ ID NO: 49; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 34 of SEQ ID NO: 5; and
   (b) a CDR2 comprising a sequence set forth in amino acids 52 to 58 of SEQ ID NO: 5; and
   (c) a CDR3 comprising a sequence set forth in amino acids 97 to 108 of SEQ ID NO: 5; and
(ii) a light chain variable region (V_{L}) comprising:
   (a) a CDR1 comprising a sequence set forth in amino acids 27 to 38 of SEQ ID NO: 6; and
   (b) a CDR2 comprising a sequence set forth in amino acids 56 to 58 of SEQ ID NO: 6; and
   (c) a CDR3 comprising a sequence set forth in amino acids 95 to 103 of SEQ ID NO: 6.

In one example, the capture protein is an antibody comprising:
(i) a heavy chain variable region (V_{H}) comprising:
   (a) a complementarity determining region (CDR) 1 comprising a sequence set forth in amino acids 26 to 34 of SEQ ID NO: 5; and
   (b) a CDR2 comprising a sequence set forth in amino acids 52 to 58 of SEQ ID NO: 5; and
   (c) a CDR3 comprising a sequence set forth in amino acids 97 to 108 of SEQ ID NO: 5; and
(ii) a light chain variable region (V_{L}) comprising:
   (a) a CDR1 comprising a sequence set forth in amino acids 27 to 38 of SEQ ID NO: 6; and
   (b) a CDR2 comprising a sequence set forth in amino acids 56 to 58 of SEQ ID NO: 6; and
   (c) a CDR3 comprising a sequence set forth in amino acids 95 to 103 of SEQ ID NO: 6.

In one example, the detection protein is an antibody comprising:
(i) a heavy chain variable region (V_{H}) comprising:
   (a) a complementarity determining region (CDR) 1 comprising a sequence set forth in amino acids 26 to 34 of SEQ ID NO: 5; and
   (b) a CDR2 comprising a sequence set forth in amino acids 52 to 58 of SEQ ID NO: 5; and
   (c) a CDR3 comprising a sequence set forth in amino acids 97 to 108 of SEQ ID NO: 5; and
(ii) a light chain variable region (V_{L}) comprising:
   (a) a CDR1 comprising a sequence set forth in amino acids 27 to 38 of SEQ ID NO: 6; and
   (b) a CDR2 comprising a sequence set forth in amino acids 56 to 58 of SEQ ID NO: 6; and
   (c) a CDR3 comprising a sequence set forth in amino acids 95 to 103 of SEQ ID NO: 6.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 7; and
   (b) a CDR2 comprising a sequence set forth in amino acids 51 to 58 of SEQ ID NO: 7; and
   (c) a CDR3 comprising a sequence set forth in amino acids 97 to 106 of SEQ ID NO: 7; and
(ii) a light chain variable region (V_{L}) comprising:
   (a) a CDR1 comprising a sequence set forth in amino acids 27 to 37 of SEQ ID NO: 8; and
   (b) a CDR2 comprising a sequence set forth in amino acids 55 to 57 of SEQ ID NO: 8; and
   (c) a CDR3 comprising a sequence set forth in amino acids 94 to 102 of SEQ ID NO: 8.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 7; and
   (b) a CDR2 comprising a sequence set forth in amino acids 51 to 58 of SEQ ID NO: 7; and
   (c) a CDR3 comprising a sequence set forth in amino acids 97 to 106 of SEQ ID NO: 7; and
(ii) a light chain variable region (V_{L}) comprising:
   (a) a CDR1 comprising a sequence set forth in amino acids 27 to 37 of SEQ ID NO: 8; and
   (b) a CDR2 comprising a sequence set forth in amino acids 55 to 57 of SEQ ID NO: 8; and
   (c) a CDR3 comprising a sequence set forth in amino acids 94 to 102 of SEQ ID NO: 8.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 7; and
   (b) a CDR2 comprising a sequence set forth in amino acids 51 to 58 of SEQ ID NO: 7; and
   (c) a CDR3 comprising a sequence set forth in amino acids 97 to 106 of SEQ ID NO: 7; and
(ii) a light chain variable region (V_{L}) comprising:
   (a) a CDR1 comprising a sequence set forth in amino acids 27 to 37 of SEQ ID NO: 8; and
   (b) a CDR2 comprising a sequence set forth in amino acids 55 to 57 of SEQ ID NO: 8; and
   (c) a CDR3 comprising a sequence set forth in amino acids 94 to 102 of SEQ ID NO: 8.

In one example, the capture protein and/or the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 52; and
   (b) a CDR2 comprising a sequence set forth in amino acids 51 to 58 of SEQ ID NO: 52; and
   (c) a CDR3 comprising a sequence set forth in amino acids 97 to 110 of SEQ ID NO: 52; and
(ii) a light chain variable region (V_{L}) comprising:
   (a) a CDR1 comprising a sequence set forth in amino acids 27 to 36 of SEQ ID NO: 53; and
   (b) a CDR2 comprising a sequence set forth in amino acids 54 to 56 of SEQ ID NO: 53; and
   (c) a CDR3 comprising a sequence set forth in amino acids 93 to 101 of SEQ ID NO: 53.

In one example, the capture protein is an antibody comprising:
(i) a heavy chain variable region (V_{H}) comprising:
   (a) a complementarity determining region (CDR) 1 comprising a sequence set forth in amino acids 26 to 33 of SEQ ID NO: 52; and
   (b) a CDR2 comprising a sequence set forth in amino acids 51 to 58 of SEQ ID NO: 52; and
   (c) a CDR3 comprising a sequence set forth in amino acids 97 to 110 of SEQ ID NO: 52; and
(ii) a light chain variable region (V_{L}) comprising:
   (a) a CDR1 comprising a sequence set forth in amino acids 27 to 36 of SEQ ID NO: 53; and
   (b) a CDR2 comprising a sequence set forth in amino acids 54 to 56 of SEQ ID NO: 53; and
   (c) a CDR3 comprising a sequence set forth in amino acids 93 to 101 of SEQ ID NO: 53.

In one example, the detection protein is an antibody comprising:
(i) a heavy chain variable region (V_{H}) comprising:
   (a) a complementarity determining region (CDR) 1 comprising a sequence set forth in amino acids 26 to 33 of SEQ ID NO: 52; and
   (b) a CDR2 comprising a sequence set forth in amino acids 51 to 58 of SEQ ID NO: 52; and
   (c) a CDR3 comprising a sequence set forth in amino acids 97 to 110 of SEQ ID NO: 52; and
(ii) a light chain variable region (V_{L}) comprising:
   (a) a CDR1 comprising a sequence set forth in amino acids 27 to 36 of SEQ ID NO: 53; and
   (b) a CDR2 comprising a sequence set forth in amino acids 54 to 56 of SEQ ID NO: 53; and
   (c) a CDR3 comprising a sequence set forth in amino acids 93 to 101 of SEQ ID NO: 53.

In one example, the capture protein and/or the detection protein is an antibody comprising:
(i) a heavy chain variable region (V_{H}) comprising:
   (a) a complementarity determining region (CDR) 1 comprising a sequence set forth in SEQ ID NO: 21, SEQ ID NO: 31 or SEQ ID NO: 37;
   (b) a CDR2 comprising a sequence set forth in SEQ ID NO: 22, SEQ ID NO: 32 or SEQ ID NO: 38; and
   (c) a CDR3 comprising a sequence set forth in SEQ ID NO: 23, SEQ ID NO: 27, SEQ ID NO: 33 or SEQ ID NO: 39; and
(ii) a light chain variable region (V_{L}) comprising:
   (a) a CDR1 comprising a sequence set forth in SEQ ID NO: 24, SEQ ID NO: 28, SEQ ID NO: 34 or SEQ ID NO: 40;
   (b) a CDR2 comprising a sequence set forth in SEQ ID NO: 25, SEQ ID NO: 29, SEQ ID NO: 35 or SEQ ID NO: 41; and
   (c) a CDR3 comprising a sequence set forth in SEQ ID NO: 26, SEQ ID NO: 30, SEQ ID NO: 36 or SEQ ID NO: 42.

In one example, the capture protein is an antibody comprising:
(i) a V_{H} comprising:
   (a) a CDR1 comprising a sequence set forth in SEQ ID NO: 21, SEQ ID NO: 31 or SEQ ID NO: 37;
   (b) a CDR2 comprising a sequence set forth in SEQ ID NO: 22, SEQ ID NO: 32 or SEQ ID NO: 38; and
   (c) a CDR3 comprising a sequence set forth in SEQ ID NO: 23, SEQ ID NO: 27, SEQ ID NO: 33 or SEQ ID NO: 39; and
(ii) a V_{L} comprising:
   (a) a CDR1 comprising a sequence set forth in SEQ ID NO: 24, SEQ ID NO: 28, SEQ ID NO: 34 or SEQ ID NO: 40;
   (b) a CDR2 comprising a sequence set forth in SEQ ID NO: 25, SEQ ID NO: 29, SEQ ID NO: 35 or SEQ ID NO: 41; and
   (c) a CDR3 comprising a sequence set forth in SEQ ID NO: 26, SEQ ID NO: 30, SEQ ID NO: 36 or SEQ ID NO: 42.

In one example, the detection protein is an antibody comprising:
(i) a V_{H} comprising:
   (a) a CDR1 comprising a sequence set forth in SEQ ID NO: 21, SEQ ID NO: 31 or SEQ ID NO: 37;
   (b) a CDR2 comprising a sequence set forth in SEQ ID NO: 22, SEQ ID NO: 32 or SEQ ID NO: 38; and
   (c) a CDR3 comprising a sequence set forth in SEQ ID NO: 23, SEQ ID NO: 27, SEQ ID NO: 33 or SEQ ID NO: 39; and
(ii) a V_{L} comprising:
   (a) a CDR1 comprising a sequence set forth in SEQ ID NO: 24, SEQ ID NO: 28, SEQ ID NO: 34 or SEQ ID NO: 40;
   (b) a CDR2 comprising a sequence set forth in SEQ ID NO: 25, SEQ ID NO: 29, SEQ ID NO: 35 or SEQ ID NO: 41; and
   (c) a CDR3 comprising a sequence set forth in SEQ ID NO: 26, SEQ ID NO: 30, SEQ ID NO: 36 or SEQ ID NO: 42.

In one example, the capture protein and/or the detection protein is an antibody comprising:
(i) a heavy chain variable region (V_{H}) comprising:
   (a) a CDR1 comprising a sequence set forth in SEQ ID NO: 21, SEQ ID NO: 31, SEQ ID NO: 37 or SEQ ID NO: 54;
   (b) a CDR2 comprising a sequence set forth in SEQ ID NO: 22, SEQ ID NO: 32, SEQ ID NO: 38 or SEQ ID NO: 55; and
   (c) a CDR3 comprising a sequence set forth in SEQ ID NO: 23, SEQ ID NO: 27, SEQ ID NO: 33, SEQ ID NO: 39 or SEQ ID NO: 56; and
(ii) a light chain variable region (V_{L}) comprising:
   (a) a CDR1 comprising a sequence set forth in SEQ ID NO: 24, SEQ ID NO: 28, SEQ ID NO: 34, SEQ ID NO: 40 or SEQ ID NO: 57;
   (b) a CDR2 comprising a sequence set forth in SEQ ID NO: 25, SEQ ID NO: 29, SEQ ID NO: 35, SEQ ID NO: 41 or SEQ ID NO: 58; and
   (c) a CDR3 comprising a sequence set forth in SEQ ID NO: 26, SEQ ID NO: 30, SEQ ID NO: 36, SEQ ID NO: 42 or SEQ ID NO: 59.

In one example, the capture protein is an antibody comprising:
(i) a V_{H} comprising:
   (a) a CDR1 comprising a sequence set forth in SEQ ID NO: 21, SEQ ID NO: 31, SEQ ID NO: 37 or SEQ ID NO: 54;
   (b) a CDR2 comprising a sequence set forth in SEQ ID NO: 22, SEQ ID NO: 32, SEQ ID NO: 38 or SEQ ID NO: 55; and
   (c) a CDR3 comprising a sequence set forth in SEQ ID NO: 23, SEQ ID NO: 27, SEQ ID NO: 33, SEQ ID NO: 39 or SEQ ID NO: 56; and
(ii) a V_{L} comprising:
   (a) a CDR1 comprising a sequence set forth in SEQ ID NO: 24, SEQ ID NO: 28, SEQ ID NO: 34, SEQ ID NO: 40 or SEQ ID NO: 57;
   (b) a CDR2 comprising a sequence set forth in SEQ ID NO: 25, SEQ ID NO: 29, SEQ ID NO: 35, SEQ ID NO: 41 or SEQ ID NO: 58; and
   (c) a CDR3 comprising a sequence set forth in SEQ ID NO: 26, SEQ ID NO: 30, SEQ ID NO: 36, SEQ ID NO: 42 or SEQ ID NO: 59.

In one example, the detection protein is an antibody comprising:
(i) a V_{H} comprising:
   (a) a CDR1 comprising a sequence set forth in SEQ ID NO: 21, SEQ ID NO: 31, SEQ ID NO: 37 or SEQ ID NO: 54;
   (b) a CDR2 comprising a sequence set forth in SEQ ID NO: 22, SEQ ID NO: 32, SEQ ID NO: 38 or SEQ ID NO: 55; and
   (c) a CDR3 comprising a sequence set forth in SEQ ID NO: 23, SEQ ID NO: 27, SEQ ID NO: 33, SEQ ID NO: 39 or SEQ ID NO: 56; and
(ii) a V_{L} comprising:
   (a) a CDR1 comprising a sequence set forth in SEQ ID NO: 24, SEQ ID NO: 28, SEQ ID NO: 34, SEQ ID NO: 40 or SEQ ID NO: 57;
   (b) a CDR2 comprising a sequence set forth in SEQ ID NO: 25, SEQ ID NO: 29, SEQ ID NO: 35, SEQ ID NO: 41 or SEQ ID NO: 58; and
   (c) a CDR3 comprising a sequence set forth in SEQ ID NO: 26, SEQ ID NO: 30, SEQ ID NO: 36, SEQ ID NO: 42 or SEQ ID NO: 59.

In one example, the capture protein and/or the detection protein is an antibody comprising:
(i) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 23; and
a V_{L} comprising:
   (a) a CDR1 comprising a sequence set forth in SEQ ID NO: 24,
   (b) a CDR2 comprising a sequence set forth in SEQ ID NO: 25, and
   (c) a CDR3 comprising a sequence set forth in SEQ ID NO: 26; or
(ii) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 27; and
a V_{L} comprising:
   (a) a CDR1 comprising a sequence set forth in SEQ ID NO: 28,
   (b) a CDR2 comprising a sequence set forth in SEQ ID NO: 29, and
   (c) a CDR3 comprising a sequence set forth in SEQ ID NO: 30; or
(iii) a V_{H} comprising:
   (a) a CDR1 comprising a sequence set forth in SEQ ID NO: 31,
   (b) a CDR2 comprising a sequence set forth in SEQ ID NO: 32, and
   (c) a CDR3 comprising a sequence set forth in SEQ ID NO: 33; and
a V_{L} comprising:
   (a) a CDR1 comprising a sequence set forth in SEQ ID NO: 34,
   (b) a CDR2 comprising a sequence set forth in SEQ ID NO: 35, and
   (c) a CDR3 comprising a sequence set forth in SEQ ID NO: 36; or
(iv) a V_{H} comprising:
   (a) a CDR1 comprising a sequence set forth in SEQ ID NO: 37,
   (b) a CDR2 comprising a sequence set forth in SEQ ID NO: 38, and
   (c) a CDR3 comprising a sequence set forth in SEQ ID NO: 39; and
a V_{L} comprising:
   (a) a CDR1 comprising a sequence set forth in SEQ ID NO: 40,
   (b) a CDR2 comprising a sequence set forth in SEQ ID NO: 41, and
   (c) a CDR3 comprising a sequence set forth in SEQ ID NO: 42.

In one example, the capture protein and/or the detection protein is an antibody comprising:
(i) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 23; and
a V_{L} comprising:
   (a) a CDR1 comprising a sequence set forth in SEQ ID NO: 24,
   (b) a CDR2 comprising a sequence set forth in SEQ ID NO: 25, and
   (c) a CDR3 comprising a sequence set forth in SEQ ID NO: 26; or
(ii) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 27; and
a V_{L} comprising:
   (a) a CDR1 comprising a sequence set forth in SEQ ID NO: 28,
   (b) a CDR2 comprising a sequence set forth in SEQ ID NO: 29, and
   (c) a CDR3 comprising a sequence set forth in SEQ ID NO: 30; or
(iii) a V_{H} comprising:
   (a) a CDR1 comprising a sequence set forth in SEQ ID NO: 31,
   (b) a CDR2 comprising a sequence set forth in SEQ ID NO: 32, and
   (c) a CDR3 comprising a sequence set forth in SEQ ID NO: 33; and
a V_{L} comprising:
   (a) a CDR1 comprising a sequence set forth in SEQ ID NO: 34,
   (b) a CDR2 comprising a sequence set forth in SEQ ID NO: 35, and
   (c) a CDR3 comprising a sequence set forth in SEQ ID NO: 36; or
(iv) a V_{H} comprising:
   (a) a CDR1 comprising a sequence set forth in SEQ ID NO: 37,
   (b) a CDR2 comprising a sequence set forth in SEQ ID NO: 38, and
   (c) a CDR3 comprising a sequence set forth in SEQ ID NO: 39; and
a V_{L} comprising:
   (a) a CDR1 comprising a sequence set forth in SEQ ID NO: 40,
   (b) a CDR2 comprising a sequence set forth in SEQ ID NO: 41, and
   (c) a CDR3 comprising a sequence set forth in SEQ ID NO: 42; or
(v) a V_{H} comprising:
   (a) a CDR1 comprising a sequence set forth in SEQ ID NO: 54,
   (b) a CDR2 comprising a sequence set forth in SEQ ID NO: 55, and
   (c) a CDR3 comprising a sequence set forth in SEQ ID NO: 56; and
a V_{L} comprising:
   (a) a CDR1 comprising a sequence set forth in SEQ ID NO: 57,
   (b) a CDR2 comprising a sequence set forth in SEQ ID NO: 58, and
   (c) a CDR3 comprising a sequence set forth in SEQ ID NO: 59.

In one example, the capture protein and/or the detection protein is an antibody comprising:
(i) a V_{H} comprising:
   (a) a CDR 1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 23; and
(ii) a V_{L} comprising:
   (a) a CDR1 comprising a sequence set forth in SEQ ID NO: 24;
   (b) a CDR2 comprising a sequence set forth in SEQ ID NO: 25; and
   (c) a CDR3 comprising a sequence set forth in SEQ ID NO: 26.

In one example, the capture protein is an antibody comprising:
(i) a V_{H} comprising:
   (a) a CDR 1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 23; and
(ii) a V_{L} comprising:
   (a) a CDR1 comprising a sequence set forth in SEQ ID NO: 24;
   (b) a CDR2 comprising a sequence set forth in SEQ ID NO: 25; and
   (c) a CDR3 comprising a sequence set forth in SEQ ID NO: 26.

In one example, the detection protein is an antibody comprising:
(i) a V_{H} comprising:
   (a) a CDR 1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 23; and
(ii) a V_{L} comprising:
   (a) a CDR1 comprising a sequence set forth in SEQ ID NO: 24;
   (b) a CDR2 comprising a sequence set forth in SEQ ID NO: 25; and
   (c) a CDR3 comprising a sequence set forth in SEQ ID NO: 26.

In one example, the capture protein and/or the detection protein is an antibody comprising:
(i) a V_{H} comprising:
   (a) a CDR 1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 27; and
(ii) a V_{L} comprising:
   (a) a CDR1 comprising a sequence set forth in SEQ ID NO: 28;
   (b) a CDR2 comprising a sequence set forth in SEQ ID NO: 29; and
   (c) a CDR3 comprising a sequence set forth in SEQ ID NO: 30.

In one example, the capture protein is an antibody comprising:
(i) a V_{H} comprising:
   (a) a CDR 1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 27; and
(ii) a V_{L} comprising:
   (a) a CDR1 comprising a sequence set forth in SEQ ID NO: 28;
   (b) a CDR2 comprising a sequence set forth in SEQ ID NO: 29; and
   (c) a CDR3 comprising a sequence set forth in SEQ ID NO: 30.

In one example, the detection protein is an antibody comprising:
(i) a V_{H} comprising:
   (a) a CDR 1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 27; and
(ii) a V_{L} comprising:
   (a) a CDR1 comprising a sequence set forth in SEQ ID NO: 28;
   (b) a CDR2 comprising a sequence set forth in SEQ ID NO: 29; and
   (c) a CDR3 comprising a sequence set forth in SEQ ID NO: 30.

In one example, the capture protein and/or the detection protein is an antibody comprising:
(i) a V_{H} comprising:
   (a) a CDR 1 comprising a sequence set forth in SEQ ID NO: 31;
   (b) a CDR2 comprising a sequence set forth in SEQ ID NO: 32; and
   (c) a CDR3 comprising a sequence set forth in SEQ ID NO: 33; and
(ii) a V_{L} comprising:
   (a) a CDR1 comprising a sequence set forth in SEQ ID NO: 34;
   (b) a CDR2 comprising a sequence set forth in SEQ ID NO: 35; and
   (c) a CDR3 comprising a sequence set forth in SEQ ID NO: 36.

In one example, the capture protein is an antibody comprising:
(i) a V_{H} comprising:
   (a) a CDR 1 comprising a sequence set forth in SEQ ID NO: 31;
   (b) a CDR2 comprising a sequence set forth in SEQ ID NO: 32; and
   (c) a CDR3 comprising a sequence set forth in SEQ ID NO: 33; and
(ii) a V_{L} comprising:
   (a) a CDR1 comprising a sequence set forth in SEQ ID NO: 34;
   (b) a CDR2 comprising a sequence set forth in SEQ ID NO: 35; and
   (c) a CDR3 comprising a sequence set forth in SEQ ID NO: 36.

In one example, the detection protein is an antibody comprising:
(i) a V_{H} comprising:
   (a) a CDR 1 comprising a sequence set forth in SEQ ID NO: 31;
   (b) a CDR2 comprising a sequence set forth in SEQ ID NO: 32; and
   (c) a CDR3 comprising a sequence set forth in SEQ ID NO: 33; and
(ii) a V_{L} comprising:
   (a) a CDR1 comprising a sequence set forth in SEQ ID NO: 34;
   (b) a CDR2 comprising a sequence set forth in SEQ ID NO: 35; and
   (c) a CDR3 comprising a sequence set forth in SEQ ID NO: 36.

In one example, the capture protein and/or the detection protein is an antibody comprising:
(i) a V_{H} comprising:
   (a) a CDR 1 comprising a sequence set forth in SEQ ID NO: 37;
   (b) a CDR2 comprising a sequence set forth in SEQ ID NO: 38; and
   (c) a CDR3 comprising a sequence set forth in SEQ ID NO: 39; and
(ii) a V_{L} comprising:
   (a) a CDR1 comprising a sequence set forth in SEQ ID NO: 40;
   (b) a CDR2 comprising a sequence set forth in SEQ ID NO: 41; and
   (c) a CDR3 comprising a sequence set forth in SEQ ID NO: 42.

In one example, the capture protein is an antibody comprising:
(i) a V_{H} comprising:
   (a) a CDR 1 comprising a sequence set forth in SEQ ID NO: 37;
   (b) a CDR2 comprising a sequence set forth in SEQ ID NO: 38; and
   (c) a CDR3 comprising a sequence set forth in SEQ ID NO: 39; and
(ii) a V_{L} comprising:
   (a) a CDR1 comprising a sequence set forth in SEQ ID NO: 40;
   (b) a CDR2 comprising a sequence set forth in SEQ ID NO: 41; and
   (c) a CDR3 comprising a sequence set forth in SEQ ID NO: 42.

In one example, the detection protein is an antibody comprising:
(i) a V_{H} comprising:
   (i) a CDR 1 comprising a sequence set forth in SEQ ID NO: 37;
   (ii) a CDR2 comprising a sequence set forth in SEQ ID NO: 38; and
   (iii) a CDR3 comprising a sequence set forth in SEQ ID NO: 39; and
(ii) a V_{L} comprising:
   (a) a CDR1 comprising a sequence set forth in SEQ ID NO: 40;
   (b) a CDR2 comprising a sequence set forth in SEQ ID NO: 41; and
   (c) a CDR3 comprising a sequence set forth in SEQ ID NO: 42.

In one example, the capture protein and/or the detection protein is an antibody comprising:
(i) a V_{H} comprising:
   (a) a CDR1 comprising a sequence set forth in SEQ ID NO: 54,
   (b) a CDR2 comprising a sequence set forth in SEQ ID NO: 55, and
   (c) a CDR3 comprising a sequence set forth in SEQ ID NO: 56; and
(ii) a V_{L} comprising:
   (a) a CDR1 comprising a sequence set forth in SEQ ID NO: 57,
   (b) a CDR2 comprising a sequence set forth in SEQ ID NO: 58, and
   (c) a CDR3 comprising a sequence set forth in SEQ ID NO: 59.

In one example, the capture protein is an antibody comprising:
(i) a V_{H} comprising:
   (a) a CDR1 comprising a sequence set forth in SEQ ID NO: 54,
   (b) a CDR2 comprising a sequence set forth in SEQ ID NO: 55, and
   (c) a CDR3 comprising a sequence set forth in SEQ ID NO: 56; and
(ii) a V_{L} comprising:
   (d) a CDR1 comprising a sequence set forth in SEQ ID NO: 57,
   (e) a CDR2 comprising a sequence set forth in SEQ ID NO: 58, and
   (f) a CDR3 comprising a sequence set forth in SEQ ID NO: 59.

In one example, the detection protein is an antibody comprising:
(i) a V_{H} comprising:
   (a) a CDR1 comprising a sequence set forth in SEQ ID NO: 54,
   (b) a CDR2 comprising a sequence set forth in SEQ ID NO: 55, and
   (c) a CDR3 comprising a sequence set forth in SEQ ID NO: 56; and
(ii) a V_{L} comprising:
   (a) a CDR1 comprising a sequence set forth in SEQ ID NO: 57,
   (b) a CDR2 comprising a sequence set forth in SEQ ID NO: 58, and
   (c) a CDR3 comprising a sequence set forth in SEQ ID NO: 59.

In one example, the capture protein and/or the detection protein is an antibody comprising:
(i) a V_{H} comprising a sequence set forth in SEQ ID NO: 1, SEQ ID NO: 3, SEQ ID NO: 5, SEQ ID NO: 7 or SEQ ID NO: 49; and
(ii) a V_{L} comprising a sequence set forth in SEQ ID NO: 2, SEQ ID NO: 4, SEQ ID NO: 6 or SEQ ID NO: 8.

In one example, the capture protein is an antibody comprising:
(i) a V_{H} comprising a sequence set forth in SEQ ID NO: 1, SEQ ID NO: 3, SEQ ID NO: 5, SEQ ID NO: 7 or SEQ ID NO: 49; and
(ii) a V_{L} comprising a sequence set forth in SEQ ID NO: 2, SEQ ID NO: 4, SEQ ID NO: 6 or SEQ ID NO: 8.

In one example, the detection protein is an antibody comprising:
(i) a V_{H} comprising a sequence set forth in SEQ ID NO: 1, SEQ ID NO: 3, SEQ ID NO: 5, SEQ ID NO: 7 or SEQ ID NO: 49; and
(ii) a V_{L} comprising a sequence set forth in SEQ ID NO: 2, SEQ ID NO: 4, SEQ ID NO: 6 or SEQ ID NO: 8.

In one example, the capture protein and/or the detection protein is an antibody comprising:
(i) a V_{H} comprising a sequence set forth in SEQ ID NO: 1, SEQ ID NO: 3, SEQ ID NO: 5, SEQ ID NO: 7, SEQ ID NO: 49 or SEQ ID NO: 52; and
(ii) a V_{L} comprising a sequence set forth in SEQ ID NO: 2, SEQ ID NO: 4, SEQ ID NO: 6, SEQ ID NO: 8 or SEQ ID NO: 53.

In one example, the capture protein is an antibody comprising:
(i) a V_{H} comprising a sequence set forth in SEQ ID NO: 1, SEQ ID NO: 3, SEQ ID NO: 5, SEQ ID NO: 7, SEQ ID NO: 49 or SEQ ID NO: 52; and
(ii) a V_{L} comprising a sequence set forth in SEQ ID NO: 2, SEQ ID NO: 4, SEQ ID NO: 6, SEQ ID NO: 8 or SEQ ID NO: 53.

In one example, the detection protein is an antibody comprising:
(i) a V_{H} comprising a sequence set forth in SEQ ID NO: 1, SEQ ID NO: 3, SEQ ID NO: 5, SEQ ID NO: 7, SEQ ID NO: 49 or SEQ ID NO: 52; and
(ii) a V_{L} comprising a sequence set forth in SEQ ID NO: 2, SEQ ID NO: 4, SEQ ID NO: 6, SEQ ID NO: 8 or SEQ ID NO: 53.

In one example, the amino acid sequence of V_{H} CDR3 comprises a consensus sequence set forth in SEQ ID NO: 49, wherein the amino acid sequence comprises Alanine (A) or Valine (V) at position 24 and/or Methionine (M) or Isoleucine (I) at position 34 and/or Cysteine (C) or Tyrosine (Y) at position 94 and/or Glycine (G) or Serine(S) at position 98 and/or Leucine (L) or Tyrosine (Y) at position 100 and/or Alanine (A) or Serine(S) at position 106 and/or Proline (P) or Tryptophan (W) at position 107.

In one example, the capture protein and/or the detection protein is an antibody comprising:
(i) a V_{H} comprising a sequence set forth in SEQ ID NO: 49; and
(ii) a V_{L} comprising a sequence set forth in SEQ ID NO: 2.

In one example, the capture protein is an antibody comprising:
(i) a V_{H} comprising a sequence set forth in SEQ ID NO: 49; and

(ii) a V_{L} comprising a sequence set forth in SEQ ID NO: 2.

In one example, the detection protein is an antibody comprising:
(i) a V_{H} comprising a sequence set forth in SEQ ID NO: 49; and
(ii) a V_{L} comprising a sequence set forth in SEQ ID NO: 2.

In one example, the amino acid sequence of V_{H} CDR3 comprises a sequence set forth in SEQ ID NO: 49, wherein the amino acid sequence comprises Alanine (A) at position 24, Isoleucine (I) at position 34, Cysteine (C) at position 94, Glycine (G) at position 98, Leucine (L) at position 100, Alanine (A) at position 106 and Proline (P) at position 107.

In one example, the capture protein and/or the detection protein is an antibody comprising:
(i) a V_{H} comprising a sequence set forth in SEQ ID NO: 49; and
(ii) a V_{L} comprising a sequence set forth in SEQ ID NO: 4.

In one example, the capture protein is an antibody comprising:
(i) a V_{H} comprising a sequence set forth in SEQ ID NO: 49; and
(ii) a V_{L} comprising a sequence set forth in SEQ ID NO: 4.

In one example, the detection protein is an antibody comprising:
(i) a V_{H} comprising a sequence set forth in SEQ ID NO: 49; and
(ii) a V_{L} comprising a sequence set forth in SEQ ID NO: 4.

In one example, the amino acid sequence of V_{H} CDR3 comprises a sequence set forth in SEQ ID NO: 49, wherein the amino acid sequence comprises Valine (V) at position 24, Methionine (M) at position 34, Tyrosine (Y) at position 94, Serine(S) at position 98, Tyrosine (Y) at position 100, Serine(S) at position 106 and Tryptophan (W) at position 107.

In one example, the capture protein and/or the detection protein is an antibody comprising:
(i) a V_{H} comprising a sequence set forth in SEQ ID NO: 1 and a V_{L} comprising a sequence set forth in SEQ ID NO: 2;
(ii) a V_{H} comprising a sequence set forth in SEQ ID NO: 3 and a V_{L} comprising a sequence set forth in SEQ ID NO: 4;
(iii) a V_{H} comprising a sequence set forth in SEQ ID NO: 5 and a V_{L} comprising a sequence set forth in SEQ ID NO: 6;
(iv) a V_{H} comprising a sequence set forth in SEQ ID NO: 7 and a V_{L} comprising a sequence set forth in SEQ ID NO: 8.

In one example, the capture protein and/or the detection protein is an antibody comprising:
(a) a V_{H} comprising a sequence set forth in SEQ ID NO: 1 and a V_{L} comprising a sequence set forth in SEQ ID NO: 2;
(b) a V_{H} comprising a sequence set forth in SEQ ID NO: 3 and a V_{L} comprising a sequence set forth in SEQ ID NO: 4;
(c) a V_{H} comprising a sequence set forth in SEQ ID NO: 5 and a V_{L} comprising a sequence set forth in SEQ ID NO: 6;
(d) a V_{L} comprising a sequence set forth in SEQ ID NO: 7 and a V_{L} comprising a sequence set forth in SEQ ID NO: 8;
(e) a V_{H} comprising a sequence set forth in SEQ ID NO: 52 and a V_{L} comprising a sequence set forth in SEQ ID NO: 53.

In one example, the capture protein and/or the detection protein is an antibody comprising a V_{H} comprising a sequence set forth in SEQ ID NO: 52 and a V_{L} comprising a sequence set forth in SEQ ID NO: 53.

In one example, the capture protein is an antibody comprising a V_{H} comprising a sequence set forth in SEQ ID NO: 52 and a V_{L} comprising a sequence set forth in SEQ ID NO: 53.

In one example, the detection protein is an antibody comprising a V_{H} comprising a sequence set forth in SEQ ID NO: 52 and a V_{L} comprising a sequence set forth in SEQ ID NO: 53.

In one example, the capture protein and/or the detection protein is an antibody comprising:
(i) a V_{H} comprising a sequence set forth in SEQ ID NO: 1; and
(ii) a V_{L} comprising a sequence set forth in SEQ ID NO: 2.

In one example, the capture protein is an antibody comprising:
(i) a V_{H} comprising a sequence set forth in SEQ ID NO: 1; and
(ii) a V_{L} comprising a sequence set forth in SEQ ID NO: 2.

In one example, the detection protein is an antibody comprising:
(i) a V_{H} comprising a sequence set forth in SEQ ID NO: 1; and
(ii) a V_{L} comprising a sequence set forth in SEQ ID NO: 2.

In one example, the capture protein and/or the detection protein is an antibody comprising:
(i) a V_{H} comprising a sequence set forth in SEQ ID NO: 3; and
(ii) a V_{L} comprising a sequence set forth in SEQ ID NO: 4.

In one example, the capture protein is an antibody comprising:
(i) a V_{H} comprising a sequence set forth in SEQ ID NO: 3; and
(ii) a V_{L} comprising a sequence set forth in SEQ ID NO: 4.

In one example, the detection protein is an antibody comprising:
(i) a V_{H} comprising a sequence set forth in SEQ ID NO: 3; and
(ii) a V_{L} comprising a sequence set forth in SEQ ID NO: 4.

In one example, the capture protein and/or the detection protein is an antibody comprising:
(i) a V_{H} comprising a sequence set forth in SEQ ID NO: 5; and
(ii) a V_{L} comprising a sequence set forth in SEQ ID NO: 6.

In one example, the capture protein is an antibody comprising:
(i) a V_{H} comprising a sequence set forth in SEQ ID NO: 5; and
(ii) a V_{L} comprising a sequence set forth in SEQ ID NO: 6.

In one example, the detection protein is an antibody comprising:
(i) a V_{H} comprising a sequence set forth in SEQ ID NO: 5; and
(ii) a V_{L} comprising a sequence set forth in SEQ ID NO: 6.

In one example, the capture protein and/or the detection protein is an antibody comprising:
(i) a V_{H} comprising a sequence set forth in SEQ ID NO: 7; and
(ii) a V_{L} comprising a sequence set forth in SEQ ID NO: 8.

In one example, the capture protein is an antibody comprising:
(i) a V_{H} comprising a sequence set forth in SEQ ID NO: 7; and
(ii) a V_{L} comprising a sequence set forth in SEQ ID NO: 8.

In one example, the detection protein is an antibody comprising:
(i) a V_{L} comprising a sequence set forth in SEQ ID NO: 7; and
(ii) a V_{L} comprising a sequence set forth in SEQ ID NO: 8.

In one example, the capture protein and the detection protein are the same. In one example, the capture protein and the detection protein are both antibodies (i.e., the same antibody).

In one example, the capture protein and the detection protein is an antibody comprising a V_{H} comprising a sequence set forth in SEQ ID NO: 1 and a V_{L} comprising a sequence set forth in SEQ ID NO: 2.

In one example, the capture protein and the detection protein is an antibody comprising a V_{H} comprising a sequence set forth in SEQ ID NO: 3 and a V_{L} comprising a sequence set forth in SEQ ID NO: 4.

In one example, the capture protein and the detection protein is an antibody comprising a V_{H} comprising a sequence set forth in SEQ ID NO: 5 and a V_{L} comprising a sequence set forth in SEQ ID NO: 6.

In one example, the capture protein and the detection protein is an antibody comprising a V_{H} comprising a sequence set forth in SEQ ID NO: 7 and a V_{L} comprising a sequence set forth in SEQ ID NO: 8.

In one example, the capture protein and the detection protein is an antibody comprising a V_{H} comprising a sequence set forth in SEQ ID NO: 52 and a V_{L} comprising a sequence set forth in SEQ ID NO: 53.

In one example, the capture protein and the detection protein are different. In one example, the capture protein and the detection protein are both antibodies but are different antibodies.

In one example, the capture protein is an antibody comprising a V_{H} comprising a sequence set forth in SEQ ID NO: 1 and a V_{L} comprising a sequence set forth in SEQ ID NO: 2; and the detection protein is an antibody comprising a V_{H} comprising a sequence set forth in SEQ ID NO: 3 and a V_{L} comprising a sequence set forth in SEQ ID NO: 4.

In one example, the capture protein is an antibody comprising a V_{H} comprising a sequence set forth in SEQ ID NO: 1 and a V_{L} comprising a sequence set forth in SEQ ID NO: 2; and the detection protein is an antibody comprising a V_{H} comprising a sequence set forth in SEQ ID NO: 5 and a V_{L} comprising a sequence set forth in SEQ ID NO: 6.

In one example, the capture protein is an antibody comprising a V_{H} comprising a sequence set forth in SEQ ID NO: 1 and a V_{L} comprising a sequence set forth in SEQ ID NO: 2; and the detection protein is an antibody comprising a V_{H} comprising a sequence set forth in SEQ ID NO: 7 and a V_{L} comprising a sequence set forth in SEQ ID NO: 8.

In one example, the detectable label is selected from the group consisting of a radiolabel, an enzyme, a fluorescent label, a luminescent label, a bioluminescent label, a magnetic label, a prosthetic group and a contrast agent.

In one example, the detectable label is a radiolabel.

In one example, the detectable label is an enzyme. For example, the enzyme is horseradish peroxidase (HRP).

In one example, the detectable label is a fluorescent label.

In one example, the detectable label is a luminescent label.

In one example, the detectable label is a bioluminescent label.

In one example, the detectable label is a magnetic label.

In one example, the detectable label is a prosthetic group.

In one example, the detectable label is a contrast agent.

The present disclosure provides an anti-SARS-COV-2 antibody, the antibody comprising any one of the following:
(i) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 23; and
a V_{L} comprising:
   (a) a CDR1 comprising a sequence set forth in SEQ ID NO: 24,
   (b) a CDR2 comprising a sequence set forth in SEQ ID NO: 25, and
   (c) a CDR3 comprising a sequence set forth in SEQ ID NO: 26; or
(ii) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 27; and
a V_{L} comprising:
   (a) a CDR1 comprising a sequence set forth in SEQ ID NO: 28,
   (b) a CDR2 comprising a sequence set forth in SEQ ID NO: 29, and
   (c) a CDR3 comprising a sequence set forth in SEQ ID NO: 30; or
(iii) a V_{H} comprising:
   (a) a CDR1 comprising a sequence set forth in SEQ ID NO: 31,
   (b) a CDR2 comprising a sequence set forth in SEQ ID NO: 32, and
   (c) a CDR3 comprising a sequence set forth in SEQ ID NO: 33; and
a V_{L} comprising:
   (a) a CDR1 comprising a sequence set forth in SEQ ID NO: 34,
   (b) a CDR2 comprising a sequence set forth in SEQ ID NO: 35, and
   (c) a CDR3 comprising a sequence set forth in SEQ ID NO: 36; or
(iv) a V_{H} comprising:
   (a) a CDR1 comprising a sequence set forth in SEQ ID NO: 37,
   (b) a CDR2 comprising a sequence set forth in SEQ ID NO: 38, and
   (c) a CDR3 comprising a sequence set forth in SEQ ID NO: 39; and
a V_{L} comprising:
   (a) a CDR1 comprising a sequence set forth in SEQ ID NO: 40,
   (b) a CDR2 comprising a sequence set forth in SEQ ID NO: 41, and
   (c) a CDR3 comprising a sequence set forth in SEQ ID NO: 42.

The present disclosure provides an anti-SARS-COV-2 antibody, the antibody comprising any one of the following:
(i) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 23; and
a V_{L} comprising:
   (a) a CDR1 comprising a sequence set forth in SEQ ID NO: 24,
   (b) a CDR2 comprising a sequence set forth in SEQ ID NO: 25, and
   (c) a CDR3 comprising a sequence set forth in SEQ ID NO: 26; or
(ii) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 27; and
a V_{L} comprising:
   (a) a CDR1 comprising a sequence set forth in SEQ ID NO: 28,
   (b) a CDR2 comprising a sequence set forth in SEQ ID NO: 29, and
   (c) a CDR3 comprising a sequence set forth in SEQ ID NO: 30; or
(iii) a V_{H} comprising:
   (a) a CDR1 comprising a sequence set forth in SEQ ID NO: 31,
   (b) a CDR2 comprising a sequence set forth in SEQ ID NO: 32, and
   (c) a CDR3 comprising a sequence set forth in SEQ ID NO: 33; and
a V_{L} comprising:
   (a) a CDR1 comprising a sequence set forth in SEQ ID NO: 34,
   (b) a CDR2 comprising a sequence set forth in SEQ ID NO: 35, and
   (c) a CDR3 comprising a sequence set forth in SEQ ID NO: 36; or
(iv) a V_{H} comprising:
   (a) a CDR1 comprising a sequence set forth in SEQ ID NO: 37,
   (b) a CDR2 comprising a sequence set forth in SEQ ID NO: 38, and
   (c) a CDR3 comprising a sequence set forth in SEQ ID NO: 39; and
a V_{L} comprising:
   (a) a CDR1 comprising a sequence set forth in SEQ ID NO: 40,
   (b) a CDR2 comprising a sequence set forth in SEQ ID NO: 41, and
   (c) a CDR3 comprising a sequence set forth in SEQ ID NO: 42 or
(v) a V_{H} comprising
   (a) a CDR1 comprising a sequence set forth in SEQ ID NO: 54,
   (b) a CDR2 comprising a sequence set forth in SEQ ID NO: 55, and
   (c) a CDR3 comprising a sequence set forth in SEQ ID NO: 56; and
a V_{L} comprising:
   (a) a CDR1 comprising a sequence set forth in SEQ ID NO: 57,
   (b) a CDR2 comprising a sequence set forth in SEQ ID NO: 58, and
   (c) a CDR3 comprising a sequence set forth in SEQ ID NO: 59.

The present disclosure provides an anti-SARS-COV-2 antibody, the antibody comprising:
(i) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 23; and
(ii) a V_{L} comprising:
   (a) a CDR1 comprising a sequence set forth in SEQ ID NO: 24,
   (b) a CDR2 comprising a sequence set forth in SEQ ID NO: 25, and
   (c) a CDR3 comprising a sequence set forth in SEQ ID NO: 26.

The present disclosure provides an anti-SARS-COV-2 antibody, the antibody comprising:
(i) a V_{H} comprising:
   (a) a CDR1 comprising a sequence set forth in SEQ ID NO: 21,
   (b) a CDR2 comprising a sequence set forth in SEQ ID NO: 22, and
   (c) a CDR3 comprising a sequence set forth in SEQ ID NO: 27; and
(ii) a V_{L} comprising:
   (a) a CDR1 comprising a sequence set forth in SEQ ID NO: 28,
   (b) a CDR2 comprising a sequence set forth in SEQ ID NO: 29, and
   (c) a CDR3 comprising a sequence set forth in SEQ ID NO: 30.

The present disclosure provides an anti-SARS-COV-2 antibody, the antibody comprising:
(i) a V_{H} comprising:
   (a) a CDR1 comprising a sequence set forth in SEQ ID NO: 31,
   (b) a CDR2 comprising a sequence set forth in SEQ ID NO: 32, and
   (c) a CDR3 comprising a sequence set forth in SEQ ID NO: 33; and
(ii) a V_{L} comprising:
   (a) a CDR1 comprising a sequence set forth in SEQ ID NO: 34,
   (b) a CDR2 comprising a sequence set forth in SEQ ID NO: 35, and
   (c) a CDR3 comprising a sequence set forth in SEQ ID NO: 36.

The present disclosure provides an anti-SARS-COV-2 antibody, the antibody comprising:
(i) a V_{H} comprising:
   (a) a CDR1 comprising a sequence set forth in SEQ ID NO: 37,
   (b) a CDR2 comprising a sequence set forth in SEQ ID NO: 38, and
   (c) a CDR3 comprising a sequence set forth in SEQ ID NO: 39; and
(ii) a V_{L} comprising:
   (a) a CDR1 comprising a sequence set forth in SEQ ID NO: 40,
   (b) a CDR2 comprising a sequence set forth in SEQ ID NO: 41, and
   (c) a CDR3 comprising a sequence set forth in SEQ ID NO: 42.

The present disclosure provides an anti-SARS-COV-2 antibody, the antibody comprising any one of the following:
(i) a V_{H} comprising a sequence set forth in SEQ ID NO: 1 and a V_{L} comprising a sequence set forth in SEQ ID NO: 2;
(ii) a V_{H} comprising a sequence set forth in SEQ ID NO: 3 and a V_{L} comprising a sequence set forth in SEQ ID NO: 4;
(iii) a V_{H} comprising a sequence set forth in SEQ ID NO: 5 and a V_{L} comprising a sequence set forth in SEQ ID NO: 6;
(iv) a V_{H} comprising a sequence set forth in SEQ ID NO: 7 and a V_{L} comprising a sequence set forth in SEQ ID NO: 8.

The present disclosure provides an anti-SARS-COV-2 antibody, the antibody comprising any one of the following:
(i) a V_{H} comprising a sequence set forth in SEQ ID NO: 1 and a V_{L} comprising a sequence set forth in SEQ ID NO: 2;
(ii) a V_{H} comprising a sequence set forth in SEQ ID NO: 3 and a V_{L} comprising a sequence set forth in SEQ ID NO: 4;
(iii) a V_{H} comprising a sequence set forth in SEQ ID NO: 5 and a V_{L} comprising a sequence set forth in SEQ ID NO: 6;
(iv) a V_{H} comprising a sequence set forth in SEQ ID NO: 7 and a V_{L} comprising a sequence set forth in SEQ ID NO: 8;
(v) a V_{H} comprising a sequence set forth in SEQ ID NO: 52 and a V_{L} comprising a sequence set forth in SEQ ID NO: 53.

The present disclosure provides an anti-SARS-COV-2 antibody, the antibody comprising a V_{H} comprising a sequence set forth in SEQ ID NO: 52 and a V_{L} comprising a sequence set forth in SEQ ID NO: 53.

In one example, the present disclosure provides an anti-SARS-COV-2 antibody, the antibody comprising a V_{H} comprising a sequence set forth in SEQ ID NO: 1 and a V_{L} comprising a sequence set forth in SEQ ID NO: 2.

In one example, the present disclosure provides an anti-SARS-COV-2 antibody, the antibody comprising a V_{H} comprising a sequence set forth in SEQ ID NO: 3 and a V_{L} comprising a sequence set forth in SEQ ID NO: 4.

In one example, the present disclosure provides an anti-SARS-COV-2 antibody, the antibody comprising a V_{H} comprising a sequence set forth in SEQ ID NO: 5 and a V_{L} comprising a sequence set forth in SEQ ID NO: 6.

In one example, the present disclosure provides an anti-SARS-COV-2 antibody, the antibody comprising a V_{H} comprising a sequence set forth in SEQ ID NO: 7 and a V_{L} comprising a sequence set forth in SEQ ID NO: 8.

In one example, the protein or antibody is any form of the protein or antibody encoded by a nucleic acid encoding any of the foregoing proteins or antibodies.

The present disclosure provides an anti-SARS-COV-2 antibody, the antibody comprising any one of the following:
(i) a V_{H} comprising a sequence expressed from or encoded by a nucleic acid comprising SEQ ID NO: 11 and a V_{L} comprising a sequence expressed from or encoded by a nucleic acid comprising SEQ ID NO: 12;
(ii) a V_{L} comprising a sequence expressed from or encoded by a nucleic acid comprising SEQ ID NO: 13 and a V_{L} comprising a sequence expressed from or encoded by a nucleic acid comprising SEQ ID NO: 14;
(iii) a V_{H} comprising a sequence expressed from or encoded by a nucleic acid comprising SEQ ID NO: 15 and a V_{L} comprising a sequence expressed from or encoded by a nucleic acid comprising SEQ ID NO: 16;
(iv) a V_{L} comprising a sequence expressed from or encoded by a nucleic acid comprising SEQ ID NO: 17 and a V_{L} comprising a sequence expressed from or encoded by a nucleic acid comprising SEQ ID NO: 18.

In one example, the disclosure provides an anti-SARS-COV-2 antibody comprising a V_{H} comprising a sequence expressed from or encoded by a nucleic acid comprising SEQ ID NO: 11 and a V_{L} comprising a sequence expressed from or encoded by a nucleic acid comprising SEQ ID NO: 12.

In one example, the disclosure provides an anti-SARS-COV-2 antibody comprising a V_{H} comprising a sequence expressed from or encoded by a nucleic acid comprising SEQ ID NO: 13 and a V_{L} comprising a sequence expressed from or encoded by a nucleic acid comprising SEQ ID NO: 14.

In one example, the disclosure provides an anti-SARS-COV-2 antibody comprising a V_{H} comprising a sequence expressed from or encoded by a nucleic acid comprising SEQ ID NO: 15 and a V_{L} comprising a sequence expressed from or encoded by a nucleic acid comprising SEQ ID NO: 16.

In one example, the disclosure provides an anti-SARS-COV-2 antibody comprising a V_{H} comprising a sequence expressed from or encoded by a nucleic acid comprising SEQ ID NO: 17 and a V_{L} comprising a sequence expressed from or encoded by a nucleic acid comprising SEQ ID NO: 18.

In one example, the disclosure provides an anti-SARS-COV-2 antibody comprising a V_{H} comprising a sequence expressed from or encoded by a nucleic acid comprising SEQ ID NO: 50 and a V_{L} comprising a sequence expressed from or encoded by a nucleic acid comprising SEQ ID NO: 51.

In one example, the present disclosure provides a polynucleotide encoding the anti-SARS-COV-2 antibody described herein. For example, the polynucleotide comprises a nucleic acid set forth in any one of SEQ ID NOs: 11 to 18.

In one example, the polynucleotide comprises:
(i) a V_{H} comprising a nucleic acid sequence set forth in SEQ ID NO: 11 and a V_{L} comprising a nucleic acid sequence set forth in SEQ ID NO: 12; or
(ii) a V_{H} comprising a nucleic acid sequence set forth in SEQ ID NO: 13 and a V_{L} comprising a nucleic acid sequence set forth in SEQ ID NO: 14; or
(iii) a V_{H} comprising a nucleic acid sequence set forth in SEQ ID NO: 15 and a V_{L} comprising a nucleic acid sequence set forth in SEQ ID NO: 16; or
(iv) a V_{H} comprising a nucleic acid sequence set forth in SEQ ID NO: 17 and a V_{L} comprising a nucleic acid sequence set forth in SEQ ID NO: 18.

In one example, the polynucleotide comprises:
(i) a V_{H} comprising a nucleic acid sequence set forth in SEQ ID NO: 11 and a V_{L} comprising a nucleic acid sequence set forth in SEQ ID NO: 12; or
(ii) a V_{H} comprising a nucleic acid sequence set forth in SEQ ID NO: 13 and a V_{L} comprising a nucleic acid sequence set forth in SEQ ID NO: 14; or
(iii) a V_{H} comprising a nucleic acid sequence set forth in SEQ ID NO: 15 and a V_{L} comprising a nucleic acid sequence set forth in SEQ ID NO: 16; or
(iv) a V_{H} comprising a nucleic acid sequence set forth in SEQ ID NO: 17 and a V_{L} comprising a nucleic acid sequence set forth in SEQ ID NO: 18; or (v a V_{H} comprising a nucleic acid sequence set forth in SEQ ID NO: 50 and a V_{L} comprising a nucleic acid sequence set forth in SEQ ID NO: 51.

In one example, the polynucleotide comprises a V_{H} comprising a nucleic acid sequence set forth in SEQ ID NO: 11 and a V_{L} comprising a nucleic acid sequence set forth in SEQ ID NO: 12.

In one example, the polynucleotide comprises a V_{H} comprising a nucleic acid sequence set forth in SEQ ID NO: 13 and a V_{L} comprising a nucleic acid sequence set forth in SEQ ID NO: 14.

In one example, the polynucleotide comprises a V_{H} comprising a nucleic acid sequence set forth in SEQ ID NO: 15 and a V_{L} comprising a nucleic acid sequence set forth in SEQ ID NO: 16.

In one example, the polynucleotide comprises a V_{H} comprising a nucleic acid sequence set forth in SEQ ID NO: 17 and a V_{L} comprising a nucleic acid sequence set forth in SEQ ID NO: 18.

In one example, the polynucleotide comprises a V_{H} comprising a nucleic acid sequence set forth in SEQ ID NO: 50 and a V_{L} comprising a nucleic acid sequence set forth in SEQ ID NO: 51.

The present disclosure also provides a kit or panel for detecting intact multimeric and/or intact trimeric SARS-COV-2 S protein in a sample, the kit or panel comprising one or more proteins or antibodies disclosed herein.

The present disclosure also provides a method for determining the suitability of a protein comprising a RBD of a SARS-COV-2 S protein to induce an immune response against SARS-COV-2, the method comprising:
(i) contacting a capture antibody immobilized onto a solid surface with the protein comprising the RBD of the SARS-COV-2 S protein to thereby form a protein complex, wherein the capture antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6; or
   (d) a V_{L} comprising a sequence set forth in SEQ ID NO: 7 and a V_{L} comprising a sequence set forth in SEQ ID NO: 8;
(ii) washing the solid surface to remove unbound protein;
(iii) contacting the protein complex with a labelled detection antibody, wherein the labelled detection antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{L} comprising a sequence set forth in SEQ ID NO: 5 and a V_{L} comprising a sequence set forth in SEQ ID NO: 6; or
   (d) a V_{H} comprising a sequence set forth in SEQ ID NO: 7 and a V_{L} comprising a sequence set forth in SEQ ID NO: 8; and
(vi) detecting the label, wherein detecting the label indicates that the protein comprising the RBD of the SARS-COV-2 S protein is multimeric and in a conformation sufficient to induce an immune response against SARS-COV-2.

The present disclosure also provides a method for determining the suitability of a protein comprising a RBD of a SARS-COV-2 S protein to induce an immune response against SARS-COV-2, the method comprising:
(i) contacting a capture antibody immobilized onto a solid surface with the protein comprising the RBD of the SARS-COV-2 S protein to thereby form a protein complex, wherein the capture antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6;
   (d) a V_{L} comprising a sequence set forth in SEQ ID NO: 7 and a V_{L} comprising a sequence set forth in SEQ ID NO: 8; or
   (e) a V_{H} comprising a sequence set forth in SEQ ID NO: 52 and a V_{L} comprising a sequence set forth in SEQ ID NO: 53
(ii) washing the solid surface to remove unbound protein;
(iii) contacting the protein complex with a labelled detection antibody, wherein the labelled detection antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6;
   (d) a V_{H} comprising a sequence set forth in SEQ ID NO: 7 and a V_{L} comprising a sequence set forth in SEQ ID NO: 8; or
   (e) a V_{H} comprising a sequence set forth in SEQ ID NO: 52 and a V_{L} comprising a sequence set forth in SEQ ID NO: 53; and
(iv) detecting the label, wherein detecting the label indicates that the protein comprising the RBD of the SARS-COV-2 S protein is multimeric and in a conformation sufficient to induce an immune response against SARS-COV-2.

The present disclosure also provides a method for determining the suitability of a protein comprising a RBD of a SARS-COV-2 S protein to induce an immune response against SARS-COV-2, the method comprising:
(i) contacting a capture antibody immobilized onto a solid surface with the protein comprising the RBD of the SARS-COV-2 S protein to thereby form a protein complex, wherein the capture antibody comprises a V_{H} comprising a sequence set forth in SEQ ID NO: 52 and a V_{L} comprising a sequence set forth in SEQ ID NO: 53;
(ii) washing the solid surface to remove unbound protein;
(iii) contacting the protein complex with a labelled detection antibody, wherein the labelled detection antibody comprises a V_{H} comprising a sequence set forth in SEQ ID NO: 52 and a V_{L} comprising a sequence set forth in SEQ ID NO: 53; and
(iv) detecting the label, wherein detecting the label indicates that the protein comprising the RBD of the SARS-COV-2 S protein is multimeric and in a conformation sufficient to induce an immune response against SARS-COV-2.

The present disclosure also provides a method for determining the suitability of a protein comprising a RBD of a SARS-COV-2 S protein to induce an immune response against SARS-COV-2, the method comprising:
(i) immobilizing a capture antibody onto a solid surface, wherein the capture antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6; or
   (d) a V_{H} comprising a sequence set forth in SEQ ID NO: 7 and a V_{L} comprising a sequence set forth in SEQ ID NO: 8;
(ii) contacting the antibody with the protein comprising the RBD of the SARS-CoV-2 S protein to thereby form a protein complex;
(iii) washing the solid surface to remove unbound protein;
(iv) contacting the protein complex with a labelled detection antibody, wherein the labelled detection antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6; or
   (d) a V_{H} comprising a sequence set forth in SEQ ID NO: 7 and a V_{L} comprising a sequence set forth in SEQ ID NO: 8; and
(v) detecting the label, wherein detecting the label indicates that the protein comprising the RBD of the SARS-COV-2 S protein is multimeric and in a conformation sufficient to induce an immune response against SARS-COV-2.

The present disclosure also provides a method for determining the suitability of a protein comprising a RBD of a SARS-COV-2 S protein to induce an immune response against SARS-COV-2, the method comprising:
(i) immobilizing a capture antibody onto a solid surface, wherein the capture antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6;
   (d) a V_{H} comprising a sequence set forth in SEQ ID NO: 7 and a V_{L} comprising a sequence set forth in SEQ ID NO: 8; or
   (e) a V_{H} comprising a sequence set forth in SEQ ID NO: 52 and a V_{L} comprising a sequence set forth in SEQ ID NO: 53
(ii) contacting the antibody with the protein comprising the RBD of the SARS-CoV-2 S protein to thereby form a protein complex;
(iii) washing the solid surface to remove unbound protein;
(iv) contacting the protein complex with a labelled detection antibody, wherein the labelled detection antibody comprises:
   (a) a V_{H} comprising a sequence set forth in SEQ ID NO: 1 and a V_{L} comprising a sequence set forth in SEQ ID NO: 2;
   (b) a V_{H} comprising a sequence set forth in SEQ ID NO: 3 and a V_{L} comprising a sequence set forth in SEQ ID NO: 4;
   (c) a V_{H} comprising a sequence set forth in SEQ ID NO: 5 and a V_{L} comprising a sequence set forth in SEQ ID NO: 6;
   (d) a V_{H} comprising a sequence set forth in SEQ ID NO: 7 and a V_{L} comprising a sequence set forth in SEQ ID NO: 8; or
   (e) a V_{H} comprising a sequence set forth in SEQ ID NO: 52 and a V_{L} comprising a sequence set forth in SEQ ID NO: 53; and
(v) detecting the label, wherein detecting the label indicates that the protein comprising the RBD of the SARS-COV-2 S protein is multimeric and in a conformation sufficient to induce an immune response against SARS-COV-2.

The present disclosure also provides a method for determining the suitability of a protein comprising a RBD of a SARS-COV-2 S protein to induce an immune response against SARS-COV-2, the method comprising:
(i) immobilizing a capture antibody onto a solid surface, wherein the capture antibody comprises a V_{H} comprising a sequence set forth in SEQ ID NO: 52 and a V_{L} comprising a sequence set forth in SEQ ID NO: 53;
(ii) contacting the antibody with the protein comprising the RBD of the SARS-CoV-2 S protein to thereby form a protein complex;
(iii) washing the solid surface to remove unbound protein;
(iv) contacting the protein complex with a labelled detection antibody, wherein the labelled detection antibody comprises a V_{H} comprising a sequence set forth in SEQ ID NO: 52 and a V_{L} comprising a sequence set forth in SEQ ID NO: 53; and
(v) detecting the label, wherein detecting the label indicates that the protein comprising the RBD of the SARS-COV-2 S protein is multimeric and in a conformation sufficient to induce an immune response against SARS-COV-2.

In one example, the capture antibody comprises a V_{H} comprising a sequence set forth in SEQ ID NO: 1 and a V_{L} comprising a sequence set forth in SEQ ID NO: 2.

In one example, the capture antibody comprises a V_{H} comprising a sequence set forth in SEQ ID NO: 3 and a V_{L} comprising a sequence set forth in SEQ ID NO: 4.

In one example, the capture antibody comprises a V_{H} comprising a sequence set forth in SEQ ID NO: 5 and a V_{L} comprising a sequence set forth in SEQ ID NO: 6.

In one example, the capture antibody comprises a V_{H} comprising a sequence set forth in SEQ ID NO: 7 and a V_{L} comprising a sequence set forth in SEQ ID NO: 8.

In one example, the capture antibody comprises a V_{H} comprising a sequence set forth in SEQ ID NO: 52 and a V_{L} comprising a sequence set forth in SEQ ID NO: 53.

In one example, the detection antibody comprises a V_{H} comprising a sequence set forth in SEQ ID NO: 1 and a V_{L} comprising a sequence set forth in SEQ ID NO: 2.

In one example, the detection antibody comprises a V_{H} comprising a sequence set forth in SEQ ID NO: 3 and a V_{L} comprising a sequence set forth in SEQ ID NO: 4.

In one example, the detection antibody comprises a V_{H} comprising a sequence set forth in SEQ ID NO: 5 and a V_{L} comprising a sequence set forth in SEQ ID NO: 6.

In one example, the detection antibody comprises a V_{H} comprising a sequence set forth in SEQ ID NO: 7 and a V_{L} comprising a sequence set forth in SEQ ID NO: 8.

In one example, the detection antibody comprises a V_{H} comprising a sequence set forth in SEQ ID NO: 52 and a V_{L} comprising a sequence set forth in SEQ ID NO: 53.

In one example, the disclosure provides a method for determining the suitability of a protein comprising a RBD of a SARS-COV-2 S protein to induce an immune response against SARS-COV-2, the method comprising:
(i) contacting a capture antibody onto a solid surface with the protein comprising the RBD of the SARS-COV-2 S protein to thereby form a protein complex, wherein the capture antibody comprises a V_{L} comprising a sequence set forth in SEQ ID NO: 1 and a V_{L} comprising a sequence set forth in SEQ ID NO: 2;
(ii) washing the solid surface to remove unbound protein; (ii)
(iii) contacting the protein complex with a labelled detection antibody, wherein the labelled detection antibody comprises a V_{L} comprising a sequence set forth in SEQ ID NO: 1 and a V_{L} comprising a sequence set forth in SEQ ID NO: 2; and
(iv) detecting the label, wherein detecting the label indicates that the protein comprising the RBD of the SARS-COV-2 S protein is multimeric and in a conformation sufficient to induce an immune response against SARS-COV-2.

In one example, the disclosure provides a method for determining the suitability of a protein comprising a RBD of a SARS-COV-2 S protein to induce an immune response against SARS-COV-2, the method comprising:
(i) immobilizing a capture antibody onto a solid surface, wherein the capture antibody comprises a V_{H} comprising a sequence set forth in SEQ ID NO: 1 and a V_{L} comprising a sequence set forth in SEQ ID NO: 2;
(ii) contacting the antibody with the protein comprising the RBD of the SARS-CoV-2 S protein to thereby form a protein complex;
(iii) washing the solid surface to remove unbound protein;
(iv) contacting the protein complex with a labelled detection antibody, wherein the labelled detection antibody comprises a V_{H} comprising a sequence set forth in SEQ ID NO: 1 and a V_{L} comprising a sequence set forth in SEQ ID NO: 2; and
(v) detecting the label, wherein detecting the label indicates that the protein comprising the RBD of the SARS-COV-2 S protein is multimeric and in a conformation sufficient to induce an immune response against SARS-COV-2.

In one example, the subject is a mammal, for example a primate, such as a human.

### BRIEF DESCRIPTION OF THE DRAWINGS

FIG. 1 is a graphical representation of capture/detection ELISA screening for suitability.

FIG. 2 is a series of graphical representations showing capture detection using capture antibodies (A) 30B8, (B) 6G6, (C) 33E10 and (D) 31G11. DEV2-DP; full fraction CSL451 antigen, approx. 20% high molecular weight species (HMWS) by SEC-HPLC and Purified Trimer; purified by SEC from DEV2-DS, containing <5% HMWS.

FIG. 3 is a series of graphical representations showing CSL451 antigen treated with 0.1% (w/v) SDS and 0.125% Zwittergent (control) for 3 Hrs at 37° C. before assessment by (A) homologous 30B8 capture/detection EIA and (B) heterologous 30B8 capture/33E10 detection ELISA. (C) SEC-HPLC analysis of CSL451 antigen treated at the same concentration of SDS demonstrated breakdown of Spike trimeric and higher order trimeric complexes to monomeric subunits.

FIG. 4 is a series of graphical representations showing capture-detection ELISA Format against stressed conditions and the presence of adjuvant or preservative. (A) Thermal stressed antigen (43 and 90° C.) and addition of preservative (Thiomersal). (B) pH stress reducing to pH 4.0 and rebuffering to pH7.0 (T=0) and pH 4.0 after 72 hrs (T=3 d). (C) Oxidative stress with the addition of H₂O₂ and reversion (T=0) and following 72 hrs (T=3 d). (D) Addition of MF59 adjuvant following mixing (T=0) and 72 hrs (T=3d)

FIG. 5 is a series of graphical representations showing independent aliquots of CSL451 DP at 0.2 mg/mL was held at; 45° C., 50° C., 55° C., 60° C., 65° C., 70° C. and 75° C. with mixing for 1 Hr. An aliquot was also held at room temperature for 1 Hr as a control. Samples were pre-treated (0.125% Zwittergent per 0.18 mg/mL antigen and held at 37° C./3 Hr) before assessment by 30B8 capture/detection EIA. (A) The average potency of 4 independent replicates was determined for each stressed sample against the control sample using 4-parameter PLA logistical fit. (B) The percentage degradation was calculated as the percentage loss relative to the potency of the control sample. The correlation between increased thermal stress and % potency recovery relative to control was assessed by linear regression and the correlation co-efficient (R) determined.

FIG. 6 is a series of graphical representations showing Binding Affinity Determination of Antibody 30B8 Against the SARS-COV-2 Sclamp Antigen showing 5 Assay Format (A), Sensorgrams (B) and Binding Kinetic Data (C)

### KEY TO SEQUENCE LISTING

SEQ ID NO: 1 heavy chain V_{H} amino acid sequence of antibody 30B8

SEQ ID NO: 2 light chain V_{L} amino acid sequence of antibody 30B8

SEQ ID NO: 3 heavy chain V_{H} amino acid sequence of antibody 39E7

SEQ ID NO: 4 light chain V_{L} amino acid sequence of antibody 39E7

SEQ ID NO: 5 heavy chain V_{H} amino acid sequence of antibody 6G6

SEQ ID NO: 6 light chain V_{L} amino acid sequence of antibody 6G6

SEQ ID NO: 7 heavy chain V_{H} amino acid sequence of antibody 33E10

SEQ ID NO: 8 light chain V_{L} amino acid sequence of antibody 33E10

SEQ ID NO: 9 heavy chain V_{H} amino acid sequence of antibody 31G11

SEQ ID NO: 10 light chain V_{L} amino acid sequence of antibody 31G11

SEQ ID NO: 11 heavy chain V_{H} nucleotide sequence of antibody 30B8

SEQ ID NO: 12 light chain V_{L} nucleotide sequence of antibody 30B8

SEQ ID NO: 13 heavy chain V_{H} nucleotide sequence of antibody 39E7

SEQ ID NO: 14 light chain V_{L} nucleotide sequence of antibody 39E7

SEQ ID NO: 15 heavy chain V_{H} nucleotide sequence of antibody 6G6

SEQ ID NO: 16 light chain V_{L} nucleotide sequence of antibody 6G6

SEQ ID NO: 17 heavy chain V_{H} nucleotide sequence of antibody 33E10

SEQ ID NO: 18 light chain V_{L} nucleotide sequence of antibody 33E10

SEQ ID NO: 19 heavy chain V_{H} nucleotide sequence of antibody 31G11

SEQ ID NO: 20 light chain V_{L} nucleotide sequence of antibody 31G11

SEQ ID NO: 21 heavy chain V_{H} CDR1 amino acid sequence of antibodies 30B8 and 39E7

SEQ ID NO: 22 heavy chain V_{H} CDR2 amino acid sequence of antibodies 30B8 and 39E7

SEQ ID NO: 23 heavy chain V_{H} CDR3 amino acid sequence of antibody 30B8

SEQ ID NO: 24 light chain V_{L} CDR1 amino acid sequence of antibody 30B8

SEQ ID NO: 25 light chain V_{L} CDR2 amino acid sequence of antibody 30B8

SEQ ID NO: 26 light chain V_{L} CDR3 amino acid sequence of antibody 30B8

SEQ ID NO: 27 heavy chain V_{H} CDR3 amino acid sequence of antibody 39E7

SEQ ID NO: 28 light chain V_{L} CDR1 amino acid sequence of antibody 39E7

SEQ ID NO: 29 light chain V_{L} CDR2 amino acid sequence of antibody 39E7

SEQ ID NO: 30 light chain V_{L} CDR3 amino acid sequence of antibody 39E7

SEQ ID NO: 31 heavy chain V_{H} CDR1 amino acid sequence of antibody 6G6

SEQ ID NO: 32 heavy chain V_{H} CDR2 amino acid sequence of antibody 6G6

SEQ ID NO: 33 heavy chain V_{H} CDR3 amino acid sequence of antibody 6G6

SEQ ID NO: 34 light chain V_{L} CDR1 amino acid sequence of antibody 6G6

SEQ ID NO: 35 light chain V_{L} CDR2 amino acid sequence of antibody 6G6

SEQ ID NO: 36 light chain V_{L} CDR3 amino acid sequence of antibody 6G6

SEQ ID NO: 37 heavy chain V_{H} CDR1 amino acid sequence of antibody 33E10

SEQ ID NO: 38 heavy chain V_{H} CDR2 amino acid sequence of antibody 33E10

SEQ ID NO: 39 heavy chain V_{H} CDR3 amino acid sequence of antibody 33E10

SEQ ID NO: 40 light chain V_{L} CDR1 amino acid sequence of antibody 33E10

SEQ ID NO: 41 light chain V_{L} CDR2 amino acid sequence of antibody 33E10

SEQ ID NO: 42 light chain V_{L} CDR3 amino acid sequence of antibody 33E10

SEQ ID NO: 43 heavy chain V_{H} CDR1 amino acid sequence of antibody 31G11

SEQ ID NO: 44 heavy chain V_{H} CDR2 amino acid sequence of antibody 31G11

SEQ ID NO: 45 heavy chain V_{H} CDR3 amino acid sequence of antibody 31G11

SEQ ID NO: 46 light chain V_{L} CDR1 amino acid sequence of antibody 31G11

SEQ ID NO: 47 light chain V_{L} CDR2 amino acid sequence of antibody 31G11

SEQ ID NO: 48 light chain V_{L} CDR3 amino acid sequence of antibody 31G11

SEQ ID NO: 49 heavy chain V_{H} amino acid consensus sequence of anti-SARS-CoV2 antibodies 30B8 and 39E7

SEQ ID NO: 50 heavy chain V_{H} nucleotide sequence of antibodies 27C8, 28B2 and 28B6

SEQ ID NO: 51 light chain V_{L} nucleotide sequence of antibodies 27C8, 28B2 and 28B6

SEQ ID NO: 52 heavy chain V_{H} amino acid sequence of antibodies 27C8, 28B2 and 28B6

SEQ ID NO: 53 light chain V_{L} amino acid sequence of antibodies 27C8, 28B2 and 28B6

SEQ ID NO: 54 heavy chain V_{H} CDR1 amino acid sequence of antibodies 27C8, 28B2 and 28B6

SEQ ID NO: 55 heavy chain V_{H} CDR2 amino acid sequence of antibodies 27C8, 28B2 and 28B6

SEQ ID NO: 56 heavy chain V_{H} CDR3 amino acid sequence of antibodies 27C8, 28B2 and 28B6

SEQ ID NO: 57 light chain V_{L} CDR1 amino acid sequence of antibodies 27C8, 28B2 and 28B6

SEQ ID NO: 58 light chain V_{L} CDR2 amino acid sequence of antibodies 27C8, 28B2 and 28B6

SEQ ID NO: 59 light chain V_{L} CDR3 amino acid sequence of antibodies 27C8, 28B2 and 28B6

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

### Selected Definitions

As used herein, the term “severe acute respiratory syndrome coronavirus 2 (SARS-COV-2)” also known as “2019 novel coronavirus (2019-nCOV)” and “human coronavirus 2019 (HCoV-19 or hCoV-19)” will be understood to refer to a strain of coronavirus that causes coronavirus disease 2019 (COVID-19).

As used herein, the term “detect” or “detecting” refers to the identification of the presence or existence of SARS-COV-2 spike protein in a sample.

As used herein, the phrase “intact” in reference to multimeric and/or trimeric protein refers to conformationally intact immunogens. The term does not include species formed as a result of cleavage of the S protein.

As used herein, the term “binds” in reference to the interaction of an antibody with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the antibody, will reduce the amount of labeled “A” bound to the antibody.

As used herein, the term “specifically binds” or “binds specifically” shall be taken to mean that an antibody of the disclosure reacts or associates more frequently, more rapidly, with greater duration and/or with greater affinity with a particular antigen (e.g., S protein or RBD) than it does with alternative antigens. For example, an antibody binds to S protein or RBD with materially greater affinity (e.g., 20 fold or 40 fold or 60 fold or 80 fold to 100 fold or 150 fold or 200 fold greater affinity) avidity, more readily, and/or with greater duration than it binds to other antigens, e.g., to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “zwittergent” or “zwitterionic detergent” will be understood to refer to a group of detergents containing a betaine and with or without a sulfonate group (i.e., sulphonated zwitterionic or non-sulphonated zwitterionic detergents); such molecules retain their amphoteric (zwitterionic) properties over a very wide range and retain the native state of the target protein without altering the charge of the protein. Exemplary non-sulphonated zwitterionic detergents include EMPIGEN BB (n-dodecyl-N,Ndimethylglycine).

As used herein, the term “non-zwittergent” or “non-zwitterionic detergent” will be understood to refer to a group of detergents with head groups that are uncharged.

As used herein, the term “non-detergent sulfobetaines” or “NDSBs” will be understood to refer to a zwitterionic-like excipient comprising a hydrophilic sulfobetaine head group and shorter hydrophobic chains than zwitterionic detergents.

The term “recombinant” shall be understood to mean the product of artificial genetic recombination. Accordingly, in the context of a recombinant antibody, this term does not encompass an antibody naturally-occurring within a subject's body that is the product of natural recombination that occurs during B cell maturation. However, if such an antibody is isolated, it is to be considered an isolated protein comprising an antibody variable region. Similarly, if nucleic acid encoding the protein is isolated and expressed using recombinant means, the resulting protein is a recombinant protein comprising an antibody variable region. A recombinant protein also encompasses a protein expressed by artificial recombinant means when it is within a cell, tissue or subject, e.g., in which it is expressed.

As used herein, the term “nucleotide sequence” or “nucleic acid sequence” will be understood to mean a series of contiguous nucleotides (or bases) covalently linked to a phosphodiester backbone.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196:901-917, 1987; Chothia et al. Nature 342, 877-883, 1989; and/or Al-Lazikani et al., J Mol Biol 273:927-948, 1997; the IMGT numbering system of Lefranc et al., Devel. And Compar. Immunol., 27:55-77, 2003; or the AHO numbering system of Honnegher and Plükthun J. Mol. Biol., 309:657-670, 2001.

“Framework regions” (FRs) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy (C_{H})1, a linker, a C_{H}2 and a C_{H}3. In a heavy chain, a constant region comprises a Fc. In a light chain, a constant region generally comprises one constant domain (a C_{L}1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG₁), gamma 2 (IgG₂) and gamma 3 (IgG₃), or hybrids thereof.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules, half antibodies and multispecific antibodies formed from antibody fragments.

As used herein, the term “subject” shall be taken to mean any animal including humans, for example a mammal. Exemplary subjects include but are not limited to humans and non-human primates. For example, the subject is a human.

### SARS-COV-2 Spike Protein

The present disclosure provides a method of detecting intact multimeric and/or intact trimeric SARS-COV-2 S protein.

SARS-COV-2 is a member of the Coronaviridae family of enveloped, positive-sense single-stranded RNA viruses. The SARS-COV-2 protein comprises four structural proteins: spike(S), membrane (M), nucleocapsid (N) and envelope (E).

The S protein is responsible for recognizing the target angiotensin converting enzyme 2 (ACE2) receptor and mediating fusion of the virus and the target cell membrane, which is considered as key to the infection process. The S protein is a large type I transmembrane protein that is highly glycosylated. It contains two subunits, S1 and S2. There are two important domains in S1 subunits, known as the N-Terminal Domain (NTD) and the Receptor Binding Domain (RBD), which is responsible for binding to ACE2. S2 has three domains called Heptad Repeat (HR), Central Helix (C_{H}), and Connector Domain (CD) respectively. Additionally, there is a furin cleavage site at S1/S2.

The S protein protrudes from the viral surface as a homotrimer with two different conformations, pre-fusion and post-fusion. It is the trimeric assembly of the S protein on the virion surface that gives it the distinctive “corona” or crown-like appearance. The binding of the RBD to ACE2 triggers the structural change from pre- to post-fusion, resulting in dissociation of the S1 and S2 subunits and transformation of the S2 subunit into a highly stable post-fusion conformation.

In one example, the method comprises detecting intact trimeric S protein. For example, the S protein is not cleaved or dissociated into S1 and S2 subunits. It will be apparent to the skilled person that the intact trimeric S protein (i.e., homotrimer) comprises a RBD on each monomeric subunit (i.e., S1 subunit) of the trimer. To facilitate a clear description of the trimeric S protein, particular components are referred to as e.g., a “first RBD” and a “second RBD”. It is to be understood that the first and second RBDs can appear in any desired order or orientation, unless otherwise specified, and that no particular order or orientation is intended by the words “first”, “second” etc.

In one example, the binding of intact trimeric S protein is 1:1 binding. For example, the capture protein binds to a first RBD on a first monomeric subunit of the trimeric S protein, the detection protein binds to a second RBD on a second monomeric subunit of the trimeric S protein and there is no binding of a third RBD on a third monomeric subunit of the trimeric S protein.

In one example, the binding of intact trimeric S protein is 1:2 binding. For example, the capture protein binds to a first RBD on a first monomeric subunit of the trimeric S protein and the detection protein binds the second and third RBDs on the second and third monomeric subunits of the trimeric S protein.

### Detecting SARS-COV-2 S Protein

The present disclosure provides a method for detecting intact multimeric and/or intact trimeric SARS-COV-2 S protein in a sample.

The skilled person will understand that the method of the present disclosure is based on a capture enzyme-linked immunosorbent assay (ELISA) or enzyme immunoassay (EIA). As used herein, the term “ELISA” or “sandwich ELISA” or “capture ELISA” or “EIA” refers to immobilizing a capture antibody (specific for the antigen) onto a solid support followed by addition of an amount of antigen. The bound antigen is then detected by a second antibody (i.e., detection antibody) which recognizes a region on the antigen that is different from that of the capture antibody. The captured antigen is detected by the detection antibody which can be covalently linked to an enzyme, or can itself be detected by addition of a secondary antibody which is linked to an enzyme.

In one example, the method of detecting intact multimeric and/or intact trimeric SARS-COV-2 S protein in a sample comprises a capture ELISA.

### Proteins Comprising Antibody Variable Regions

The present disclosure provides a capture protein comprising an antibody variable region. The present disclosure also provides a detection protein comprising an antibody variable region.

In one example, the antibody variable region binds specifically to a S protein RBD. For example, the antibody variable region of the capture protein binds a first S protein RBD and the antibody variable region of the detection protein binds to a second S protein RBD.

### Antibodies

The present disclosure provides an anti-SARS-COV-2 antibody, e.g., comprising the variable regions described herein. For example, the disclosure provides an anti-SARS-COV-2 capture antibody. The disclosure also provides an anti-SARS-COV-2 detection antibody.

In one example, the antibody is a recombinant antibody. For example, an antibody or protein comprising a variable region thereof is produced using a standard method, e.g., as is known in the art or briefly described herein.

Monoclonal antibodies are exemplary antibodies contemplated by the present disclosure. The term “monoclonal antibody” or “mAb” or “MAb” refers to a homogeneous antibody population capable of binding to the same antigen(s) and, for example, to the same epitope within the antigen. This term is not intended to be limited with respect to the source of the antibody or the manner in which it is made.

### Deimmunized, Chimeric, Humanized, Synhumanized, Primatized and Human Proteins

The proteins of the present disclosure may be a humanized protein.

The term “humanized protein” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized proteins also include proteins in which one or more residues of the human protein are modified by one or more amino acid substitutions and/or one or more FR residues of the human protein are replaced by corresponding non-human residues. Humanized proteins may also comprise residues which are found in neither the human antibody or in the non-human antibody. Any additional regions of the protein (e.g., Fc region) are generally human. Humanization can be performed using a method known in the art, e.g., U.S. Pat. Nos. 5,225,539, 6,054,297, 7,566,771 or U.S. Pat. No. 5,585,089. The term “humanized protein” also encompasses a super-humanized protein, e.g., as described in U.S. Pat. No. 7,732,578.

The proteins of the present disclosure may be human proteins. The term “human protein” as used herein refers to proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

The proteins of the present disclosure may be synhumanized proteins. The term “synhumanized protein” refers to a protein prepared by a method described in WO2007/019620. A synhumanized protein includes a variable region of an antibody, wherein the variable region comprises FRs from a New World primate antibody variable region and CDRs from a non-New World primate antibody variable region. For example, a synhumanized protein includes a variable region of an antibody, wherein the variable region comprises FRs from a New World primate antibody variable region and CDRs from a mouse or rat antibody.

The proteins of the present disclosure may be primatized proteins. A “primatized protein” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example a protein of the disclosure is a chimeric protein. The term “chimeric proteins” refers to proteins in which an antigen binding domain is from a particular species (e.g., murine, such as mouse or rat) or belonging to a particular antibody class or subclass, while the remainder of the protein is from a protein derived from another species (such as, for example, human or non-human primate) or belonging to another antibody class or subclass. In one example, a chimeric protein is a chimeric antibody comprising a V_{H} and/or a V_{L} from a non-human antibody (e.g., a murine antibody) and the remaining regions of the antibody are from a human antibody. The production of such chimeric proteins is known in the art, and may be achieved by standard means (as described, e.g., in U.S. Pat. Nos. 6,331,415; 5,807,715; 4,816,567 and 4,816,397).

The present disclosure also contemplates a deimmunized protein, e.g., as described in WO2000/34317 and WO2004/108158. De-immunized antibodies and proteins have one or more epitopes, e.g., B cell epitopes or T cell epitopes removed (i.e., mutated) to thereby reduce the likelihood that a subject will raise an immune response against the antibody or protein.

### Antibody Fragments

Single Chain Fv (scFv) Fragments and Dimeric-scFv (Di-scFv)

The skilled artisan will be aware that scFvs comprise V_{H} and V_{L} regions in a single polypeptide chain. The polypeptide chain further comprises a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favoured linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv (see, for example, Brinkmann et al., 1993).

Alternatively, or in addition, the present disclosure provides a dimeric scFv, i.e., a protein comprising two scFv molecules linked by a non-covalent or covalent linkage, e.g., by a leucine zipper domain (e.g., derived from Fos or Jun) (see, for example, Kruif and Logtenberg, 1996). Alternatively, two scFvs are linked by a peptide linker of sufficient length to permit both scFvs to form and to bind to an antigen, e.g., as described in US20060263367.

For a review of scFv, see Plückthun (1994).

Diabodies, Triabodies, Tetrabodies Exemplary proteins comprising an antibody antigen binding domain are diabodies, triabodies, tetrabodies and higher order protein complexes such as those described in WO98/044001 and WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form an Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

### Minibodies

The skilled artisan will be aware that a minibody comprises the V_{H} and V_{L} domains of an antibody fused to the C_{H}2 and/or C_{H}3 domain of an antibody. Optionally, the minibody comprises a hinge region between the V_{H} and a V_{L}, sometimes this conformation is referred to as a Flex Minibody. A minibody does not comprise a C_{H}1 or a C_{L}. In one example, the V_{H} and V_{L} domains are fused to the hinge region and the C_{H}3 domain of an antibody. At least one of the variable regions of said minibody binds to the S protein RBD in the manner of the disclosure. Exemplary minibodies and methods for their production are described, for example, in WO94/09817.

Constant Domain Fusions The present disclosure encompasses proteins comprising a variable region and a constant region or a domain(s) thereof, e.g., Fc, C_{H}2 and/or C_{H}3 domain. The skilled artisan will be aware of the meaning of the terms constant region and constant domain based on the disclosure herein and references discussed herein.

Constant region sequences useful for producing the proteins of the present disclosure may be obtained from a number of different sources. In some examples, the constant region or portion thereof of the protein is derived from a human antibody. Moreover, the constant domain or portion thereof may be derived from any antibody class, including IgM, IgG, IgD, IgA and IgE, and any antibody isotype, including IgG₁, IgG₂, IgG₃ and IgG₄.

A variety of constant region gene sequences are available in the form of publicly accessible deposits or the sequence thereof is available from publicly available databases. Constant regions can be selected having a particular effector function (or lacking a particular effector function) or with a particular modification to reduce immunogenicity.

The present disclosure also contemplates proteins comprising mutant constant regions or domains, e.g., as described in U.S. Pat. Nos. 7,217,797; 7,217,798; or US20090041770 (having increased half-life) or US2005037000 (increased ADCC).

### Protein Production

In one example, a protein or antibody of the disclosure is produced by culturing a cell line under conditions sufficient to produce the protein, e.g., as described herein and/or as is known in the art.

### Recombinant Expression

In the case of a recombinant protein, nucleic acid encoding same is placed into one or more expression construct, e.g., expression vector(s), which is/are then transfected into host cells, such as cells that can produce a disulphide bridge or bond, such as E. coli cells, yeast cells, insect cells, or mammalian cells. Exemplary mammalian cells include simian COS cells, Chinese Hamster Ovary (CHO) cells, or myeloma cells that do not otherwise produce immunoglobulin protein. Molecular cloning techniques to achieve these ends are known in the art and described, for example in Ausubel or Sambrook. A wide variety of cloning and in vitro amplification methods are suitable for the construction of recombinant nucleic acids. Methods of producing recombinant antibodies are also known in the art. See U.S. Pat. Nos. 4,816,567; 7,923,221 and 7,022,500.

Following isolation, the nucleic acid encoding a protein of the disclosure is inserted into an expression construct or replicable vector for further cloning (amplification of the DNA) or for expression in a cell-free system or in cells. For example, the nucleic acid is operably linked to a promoter,

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein of the present disclosure (e.g., derived from the information provided herein), an enhancer element, a promoter, and a transcription termination sequence. The skilled artisan will be aware of suitable sequences for expression of a protein. For example, exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters include those active in prokaryotes (e.g., phoA promoter, β-lactamase and lactose promoter systems, alkaline phosphatase, a tryptophan (trp) promoter system, and hybrid promoters such as the tac promoter).

Exemplary promoters active in mammalian cells include cytomegalovirus immediate early promoter (CMV-IE), human elongation factor 1-αpromoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CVI line transformed by SV40 (COS-7, AUSTRALIAN CELL BANK CRL 1651); human embryonic kidney line (293 or 293 cells subcloned for growth in suspension culture; baby hamster kidney cells (BHK, AUSTRALIAN CELL BANK CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid molecule or a gene construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation, viral transduction (e.g., using a lentivirus) and microparticle bombardment such as by using DNA-coated tungsten or gold particles (Agracetus Inc., WI, USA) amongst others.

The host cells used to produce the protein of the disclosure may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

A protein or antibody of the present disclosure can be isolated or purified.

Methods for purifying a protein or antibody of the disclosure are known in the art and/or described herein.

When using recombinant techniques, the protein or antibody of the disclosure can be produced intracellularly, in the periplasmic space, or directly secreted into the medium. If the protein is produced intracellularly, as a first step, the particulate debris, either host cells or lysed fragments, is removed, for example, by centrifugation or ultrafiltration. Where the protein is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Zola (1997).

The skilled artisan will also be aware that a protein of the disclosure can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. For example, the tag is a hexa-his tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Conjugates

The present disclosure also provides conjugates of proteins described herein according to any example. For example, a protein comprising an antibody variable region is conjugated to a detectable label.

As used herein, the term “conjugate” or “conjugated” shall be understood to encompass both indirect and direct binding. For example, direct conjugation includes chemical conjugation, which can be non-covalent or covalent or genetic conjugation (also referred to as “fusion”). In one example, the conjugation is covalent, e.g., a disulphide bond.

As used herein, a “detectable label” is a molecular or atomic tag or marker that generates or can be induced to generate an optical or other signal or product that can be detected visually or by using a suitable detector. Detectable labels are well known in the art and include, for example, a radiolabel, an enzyme, a fluorescent label, a luminescent label, a bioluminescent label, a magnetic label, a prosthetic group, a contrast agent and an ultrasound agent.

Fluorescent labels commonly used include Alexa, cyanine such as Cy5 and Cy5.5, and indocyanine, and fluorescein isothiocyanate (FITC), but they are not so limited. Fluorescent labels useful in the practice of the present disclosure can include, also without limitation, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6C; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange+DNA; Acridine Orange+RNA; Acridine Orange, both DNA & RNA; Acridine Red; Acridine Yellow; Acriflavin; Acriflavin Feulgen SITSA; Aequorin (Photoprotein); Alexa Fluor 350; Alexa Fluor 430; Alexa Fluor 488; Alexa Fluor 532; Alexa Fluor 546; Alexa Fluor 568; Alexa Fluor 594; Alexa Fluor 633; Alexa Fluor 647; Alexa Fluor 660; Alexa Fluor 680; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Anthrocyl Blue; stearate; APC (Allophycocyanin); APC-Cy7; APTRA-BTC=Ratio Dye, Zn²⁺; APTS; Astrazon Brilliant Red 4G; Astrazon Orange R; Astrazon Red 6B; Astrazon Yellow 7 GLL; Atabrine; ATTO-TAG CBQCA; ATTO-TAG FQ; Auramine; Aurophosphine G; Aurophosphine; BAO 9 (Bisamninophenyloxadiazole); BCECF (high pH); BCECF (low pH); Berberine Sulphate; Beta Lactamase; BFP blue shifted GFP (Y66H); Blue Fluorescent Protein; BFP/GFP FRET Bimane; Bisbenzamnide; Bisbenzimide (Hoechst); bis-BTC=Ratio Dye, Zn²⁺; Blancophor FFG; Blancophor SV; BOBO-1; BOBO-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO-1; BO-PRO-3; Brilliant Sulphoflavin FF; BTC-Ratio Dye Ca²⁺; BTC-5N-atio Dye, Zn²⁺; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue; Cascade Yellow 399; Catecholamine; CCF2 (GeneBlazer); CFDA; CFP—Cyan Fluorescent Protein; CFP/YFP; FRET; Chlorophyll; Chromomycin A; Chromomycin A; CL-NERF (Ratio Dye, pH); CMFDA; Coelenterazine; Coelenterazine cp (Ca²⁺ Dye); Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2; Cy3.1 8; Cy3.5; Cy3; Cy5.1 8; Cy5.5; Cy5; Cy7; Cyan GFP; cyclic AMP Fluorosensor (FiCRhR); CyQuant Cell Proliferation Assay; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DIR; DIR (DiIC18(7)); DM-NERF (high pH); DNP; Dopamine; DsRed; Red fluorescent protein; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyde Induced Fluorescence); FITC; FITC Antibody; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43; FM 4-46; Fura Red (high pH); Fura Red/Fluo-3; Fura-2, high calcium; Fura-2, low calcium; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP), GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; LIVE/DEAD Kit Animal Cells, Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue, LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green; Oregon Green 488-X; Oregon Green; Oregon Green 488; Oregon Green 500; Oregon Greene 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodide (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI, Serotonin; Sevron Brilliant Red 2B; Sevron Brilliant Red 4G; Sevron Brilliant Red B; Sevron Orange; Sevron Yellow L; sgBFP; sgBFP (super glow BFP); sgGFP; sgGFP (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl) quinolinium); Stilbene; Sulphorhodamine B can C; Sulphorhodamine G Extra; SYTO 11; SYTO 12; SYTO 13; SYTO 14; SYTO 15; SYT; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamine-IsoThioCyanate); True Blue; TruRed; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO-3; YOYO-1; and YOYO-3.

In one example, a detectable label is an enzyme. Examples of enzymes useful in the disclosure include, without limitation, alkaline phosphatase and horseradish peroxidase. Alternatively or in addition, the enzyme can be, for example, luciferase. The enzyme can be linked to the antibody by conventional chemical methods, or it can be expressed together with the antibody as a fusion protein. In one example, the enzyme is horseradish peroxidase.

Radioisotopes useful as detectable labels in the disclosure are well known in the art and can include ³H, ¹¹C, ¹⁸F, ³⁵S, ⁶⁴Cu, ⁶⁷Ga, ⁶⁸Ga, ⁹⁹mTc, ¹¹¹In, ¹²³I, ¹²⁴I, ¹²⁵I, and ¹³¹I.

### Panels or Kits

Another example of the disclosure provides kits containing proteins or antibodies of the disclosure for use in any method described herein (e.g., detecting intact multimeric and/or intact trimeric SARS-COV-2 S protein).

In one example, the panel or kit as described herein is for ex vivo analysis. In one example, the kit is suitable for use with whole blood, plasma, serum and/or sputum samples.

In one example, the panel or kit as described herein is suitable for high-throughput screening. The term “high-throughput screening” refers to screening methods that can be used to test or assess more than one sample at a time and that can reduce the time for testing multiple samples. In one example, the methods are suitable for testing or assessing at least 5 samples, at least 10, at least 20, at least 30, at least 50, at least 70, at least 90, at least 150, at least 200, at least 300 samples at a time. Such high-throughput screening methods can analyse more than one sample rapidly e.g. in at least 30 minutes, in at least 1 hour, in at least 2 hours, in at least 3 hours, in at least 4 hours, in at least 5 hours, in at least 6 hours, in at least 7 hours, in at least 8 hours, in at least 9 hours or in at least 10 hours. High-throughput screening may also involve the use of liquid handling devices. In one example, high-throughput analysis may be automated.

The present disclosure includes the following non-limiting examples.

### EXAMPLES

### Example 1: Development of the Monoclonal Antibodies

Monoclonal antibodies against acute respiratory syndrome coronavirus 2 (SARS-CoV-2) spike protein were produced by immunising Balb/c mice with 10 μg of HEK293 SARS-COV2 S Protein (S1 Subunit, His Tag (Sino Biological) mixed with an equal volume of complete Fruends' adjuvant. Popliteal lymph nodes were harvested from Balb/c immunized mice and the lymphocytes were fused and selected as previously described (Pietrzykowski et al., Antibody Responses to HPV6b E Polyproteins and Production of Monoclonal Antibodies Hybridoma and Hybridomics. 2002) to generate hybrdoma cell lines. The hybridoma cells were cloned and screened using 4 ng/ml SARS-COV2 Spike Protein (S1 Subunit, RBD His Tag expressed in Baculovirus (Sino Biological) using a solid phase ELISA. The use of a mammalian expressed RBD containing protein followed by screening with a Baculovirus RBD protein enabled the identification of antibodies that bound SARS COV2 RBD but were refractory to the differences in glycosylation profile expressed by either baculovirus through to mammalian systems.

Confirmation of the reactivity to mammalian expressed RBD was achieved by subsequent screening of the antibody secreting hybridomas with a 2019 nCOV S protein RBD, Fc Tag (Acro Biosystems) using solid phase ELISA. Isotype determinations (Heavy/light chains) for each cloned monoclonal antibody was determined using a Roche IsoStrip Mouse Monoclonal Antibody Isotyping Kit (Merck 11493027001) as per manufacturer's instructions.

### Example 2: Screening of Antibody Candidates in Capture-Detection ELISA

Monoclonal antibodies are an antibody derived from a single B cell and therefore recognise a single epitope on a protein. For an antigen such as a homotrimer, that is a macromolecular complex formed by three identical proteins that are usually non-covalently bound there are most often three protein epitopes. To function in this ELISA format, the binding sites of two RBDs on adjacent monomer units of a trimer must be available in an orientation in which the capture and detection antibodies can both bind without sterically hindering one another.

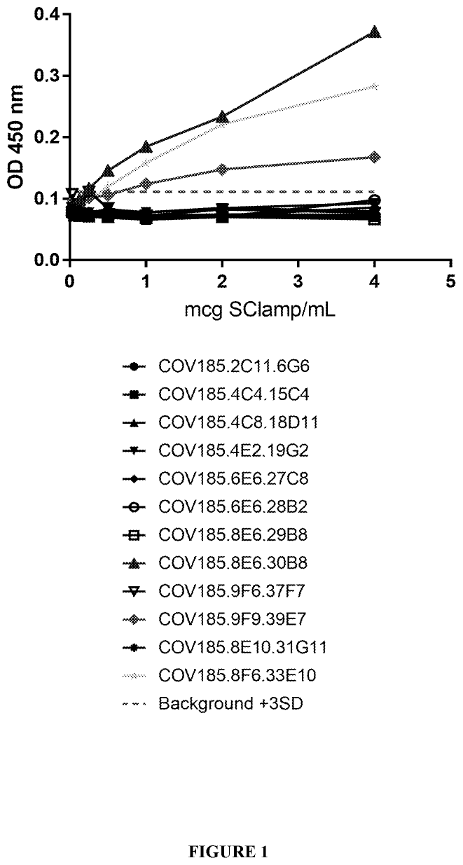

In order to determine the suitability of the monoclonal antibodies for the quantitation of SARS CoV2 protein antigen, the monoclonal antibodies were conjugated with reporter molecule; Horse Radish Peroxidase (HRP). Briefly, clonal supernatants were collected and concentrated, micro-conjugated and tested in a capture-detection ELISA using S clamp antigen from CSL451 GMP batch TC20-02-08 (sublot VS20200615A). Unconjugated monoclonal antibodies were coated onto 96 well plates (NUNC MaxiSorp™ CAT #NUN439454) at 0.14 mcg per well in Dulbecco's PBS w/o Ca+& Mg+ (Sigma, CAT #MFCD00131855), overnight at 2-8° C. S clamp Antigen was titrated down the plate at concentrations appropriate for sigmoidal modelling before incubation with HRP conjugated mAbs of the same clone. Unbound antigen was rinsed off before development of the peroxidase colorimetric reaction with TMB substrate (SeraCare, CAT #5120-0050). Colorimetric reaction was fixed with 0.55M Sulphuric acid before spectrophotometric reading at 450 nm. Not all antibodies were functional in the capture-detection ELISA screen. Examples of a non-optimised capture/detection ELISA are shown in FIG. 1. A final shortlist of four candidate antibodies (6G6, 39E7, 33E10 and 30B8) were found to be suitable for use in a homologous capture-detection ELISA format following increasing the level of available monoclonal antibody for the assay.

### Example 3. Evaluation of Candidate Antibodies in Homologous and Non-Homologous Capture-Detection ELISA

Monoclonal antibodies were set up for homologous and non-homologous capture/detection to evaluate the suitability of each monoclonal antibody. The S clamp SARS COV2 protein expressed three RBDs. Thus capturing with a single monoclonal antibody and detecting with another can allow either non-detection (interference with binding), 1:1 binding where one antibody captures and the detection occurs through binding another of one of the two RBDs but does not allow detection or binding of the third; 1:2 where one RBD is captured leaving the other two available for binding or 1:3 where the RBD is captured leaving all three RBDs available for binding and therefore complete non-interference with binding.

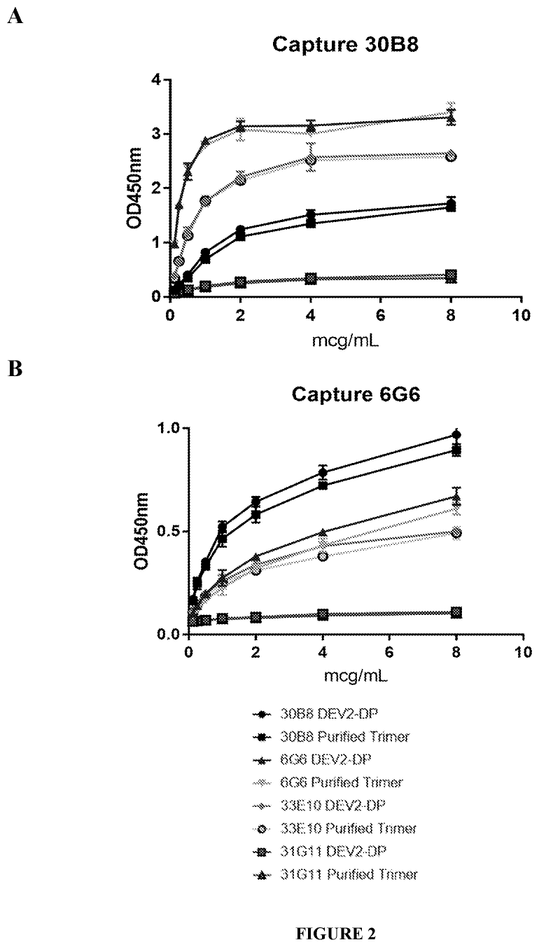

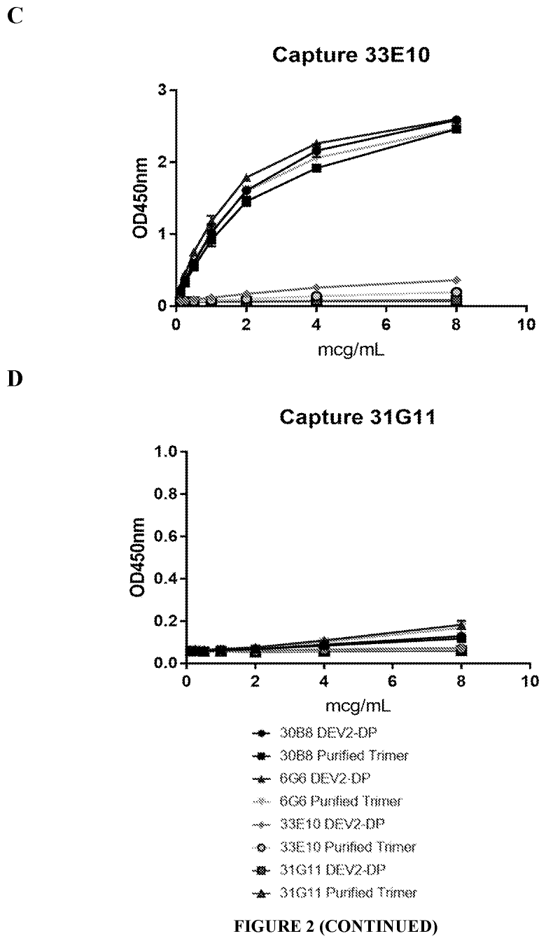

FIG. 2A illustrates each of these reactions with capturing with 30B8 and detection with 31G11 (interference and non-detection), detection with 30B8 (1:1 reactivity); detection with 33E10 (1:2 reactivity) and detection with 6G6 (1:3 reactivity). Analysis of capture with 6G6 (FIG. 2B) confirmed the 3:1 signal but was weaker in this assay, capture detection with 6G6 2:1 was similar to capture with 6G6 and detection with 33E10 in a 2:1 reactivity. These observations were confirmed when capturing with 33E10 (FIG. 2C), with 30B8 and 6G6 reacting at the same levels (2:1) whereas 31G11 (FIG. 2D) could not capture or detect in any format indicating steric hindrance of the other monoclonal antibodies to detect RBD binding.

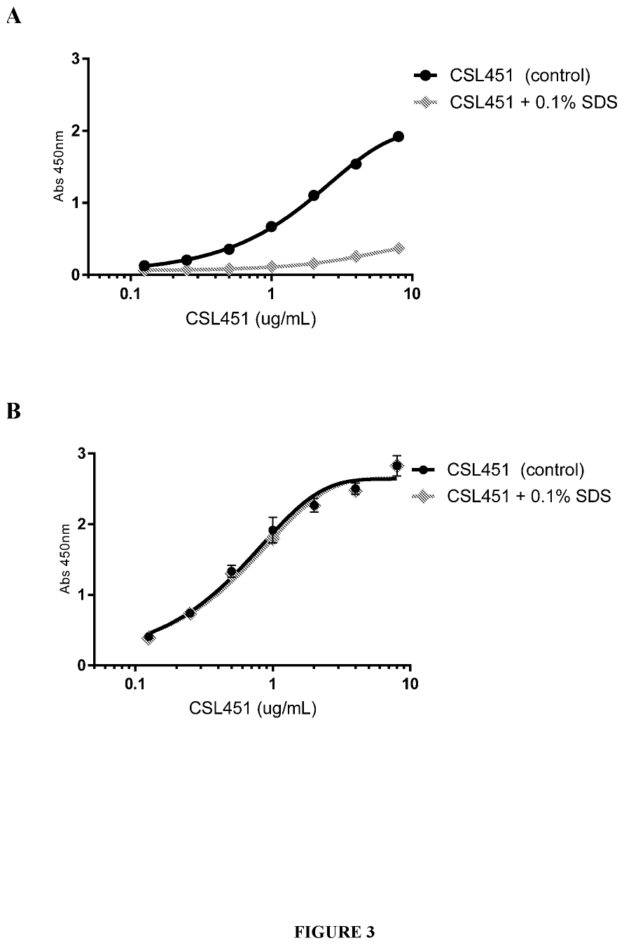

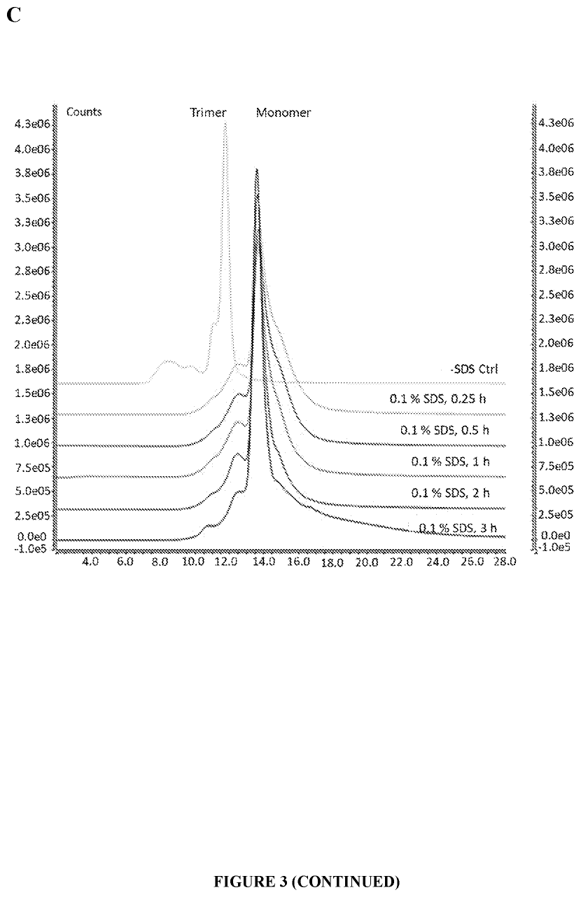

To confirm the suitability of a 30B8 capture/detection monoclonal antibody ELISA, trimeric S clamp SARS CoV2 protein was treated with different detergents (0.1% SDS or 0.125% Zwittergent (as control) to render the trimeric material to monomeric. 30B8 capture/detection ELISA indicated an expected dose response when material was treated with the control detergent (Zwittergent) (FIG. 3A). Upon treatment of this antigen (SARS CoV2 Sclamp) with 0.1% SDS, the material could no longer be detected in this capture/detection format. Analysis of these detergent treated proteins with a non-homologous set of antibodies capture (30B8) detection (33E10), both treated antigens could equally be detected (FIG. 3B). Size exclusion chromatography confirmed that following 0.1% SDS treatment, the antigen was rendered almost entirely to monomeric form compared to no-treated antigen that the majority of the material is trimeric or multimeric (FIG. 3C). This confirmed the capture/detection ELISA with 30B8 to detect intact trimeric and high order species.

### Example 4: Evaluation of Candidate Antibodies for Stability Indicating Potential in Capture-Detection ELISA Format

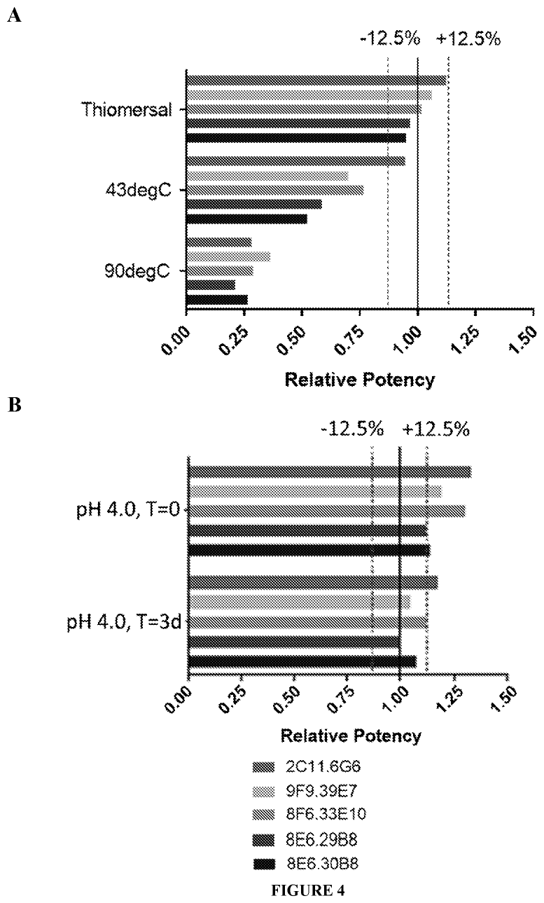

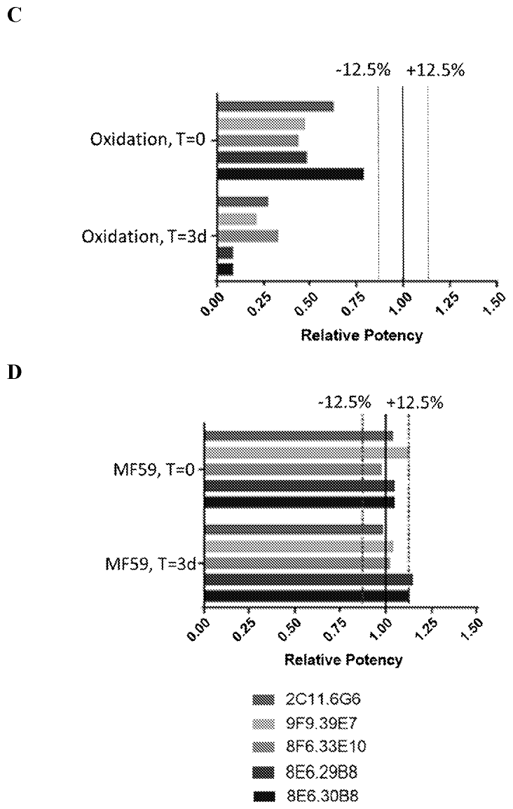

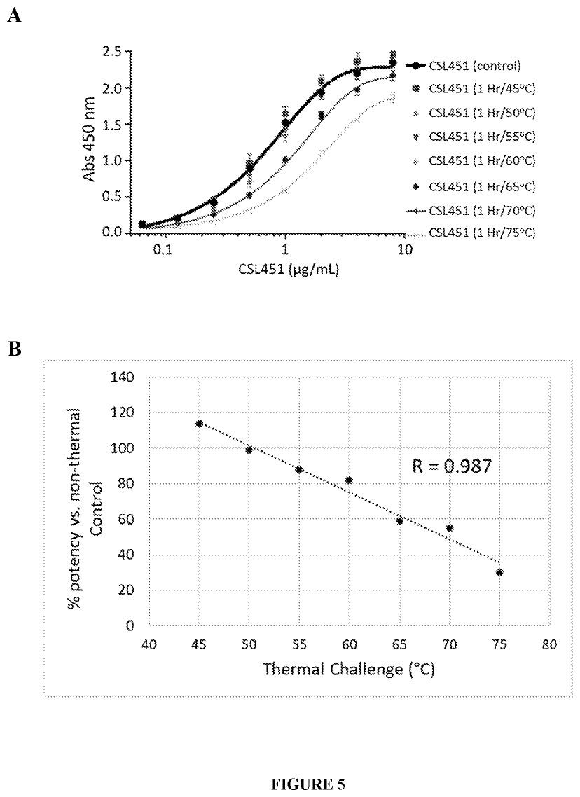

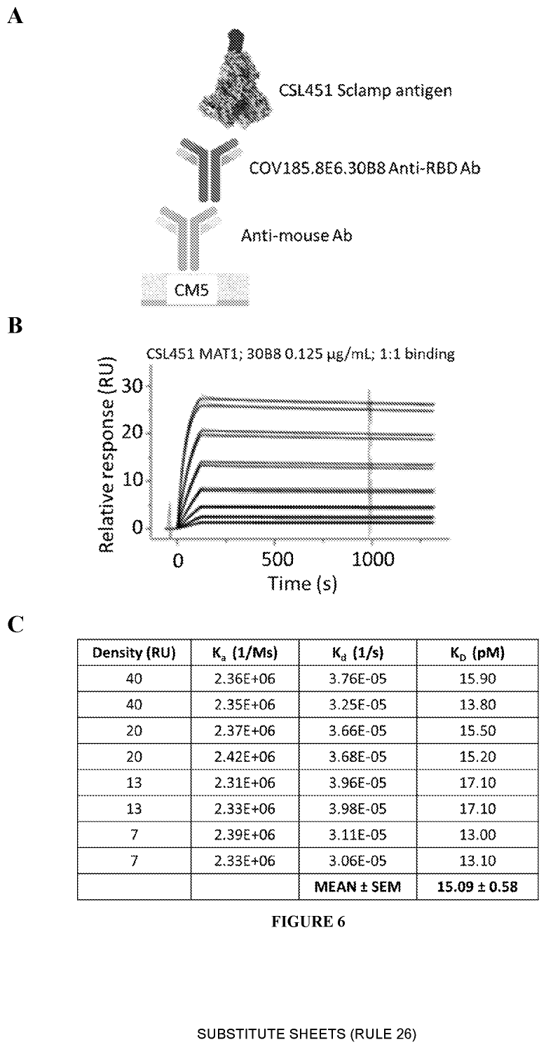

The five shortlisted antibodies were tested in a capture-detection ELISA using thermally treated S clamp antigen to test for the ability to be stability indicating and discriminate degraded or denatured antigen (FIGS. 4A and 5). All antibodies showed a decrease in response to some degree against material treated with either 43° C. and 90° C., with antibody 30B8 appearing to show the greatest sensitivity to these treatments. A titratable effect of temperature on capture/detection ELISA by 30B8 was demonstrated to be linear from 45° C. to 75° C. Altering the pH had no effect upon the antigen with any of the capture detection Mabs at time 0 or after 72 hrs (FIG. 4B). Treatment with oxidation by applying 27% (v/v) H₂O₂ decreased the level of intact antigen at time 30 minutes and was further decreased at 72 hrs (FIG. 4C). Addition of the preservative, Thiomersal (FIG. 4A—Redone FIG. 6A to be provided) and adjuvant, MF59 (FIG. 4D) did not affect the quantitation of antigen.

### Example 5: Antibody 30B8 Binds with High Efficiency and Affinity to S Clamp Antigen

Antibody 30B8 was also characterized for its binding affinity to CSL451 S clamp antigen and found to bind with a very high affinity of ˜15 pM (FIG. 6).

TABLE 3
Table of sequences

| SEQ ID | Molecule | |
|---|---|---|
| NO | type | Residues |
| 1 | Protein | EVQLQQSGAELVKPGASVKLSCTASGFNIKDTYIHWVKQRPE |
| | | QGLEWIGRIDPANGNTKYDPKFQGKATITADTSSNTAYLQLS |
| | | SLTSEDTAVCYCAGSLYDYDGAPFAYWGQGTLVTVSA |
| 2 | Protein | SIVMTQTPKFLLVSAGDRVTITCKASQSVINDVTWYQQKPG |
| | | QSPKLLIYYASNRYTGVPDRFTGSGYGTDFTFTISTVQAEDLA |
| | | VYFCQQDYSSPWTFGGGTKLEIK |
| 3 | Protein | EVQLQQSGAELVKPGASVKLSCTVSGFNIKDTYMHWVKQRP |
| | | EQGLEWIGRIDPANGNTKYDPKFQGKATITADTSSNTAYLQL |
| | | SSLTSEDTAVYYCASSYYDYDGSWFAYWGQGTLVTVSA |
| 4 | Protein | ENVLTQSPAIMSASPGEKVTMTCSASSSVSYMHWYQQKSSTS |
| | | PKIWIYDTSKLASGVPGRFSGSGSGNSYSLTISSMEAEDVATY |
| | | YCFQGSGYPLTFGSGTRLEIK |
| 5 | Protein | DVQLQESGPGLVKPSQSLSLTCTVTGYSITSDYAWNWIRQFP |
| | | GNKLEWMGYISYSGSTSYNPSLKSRISITRDTSKNQFFLQLKS |
| | | VTTEDAATYYCARGDGYFYAMDYWGQGTSVTVSS |
| 6 | Protein | DIVMTQSPSSLSVSAGEKVTMSCKSSQSLLNSGNQKSYLAWY |
| | | QQKPGQPPKLLIYGASTRESGVPDRFTGSGSGTDFTLTISSVQ |
| | | AEDLAVYSCONDHSYPPTFGAGTKLDLK |
| 7 | Protein | QVQLQQSGPQLVRPGASVKISCKASGYSFTNNWMHWVKQR |
| | | PGQGLEWIGMIDPSDSETRLNQKFKDKATLTVDKSSSTAYMQ |
| | | LSSPTSEDSAVYYCARVNWEIFDYWGQGTTLTVSS |
| 8 | Protein | DIVMTQAAPSVPVTPGESVSISCRSSKSLLNRNGNTYLYWFL |
| | | QRPGQSPQLLIYRMSNLASGVPDRFSGSGSGTAFTLRISRVEA |
| | | EDVGVYYCMQHLEYPLTFGAGTKLELK |
| 9 | Protein | QVQLQQPGAELVRPGASVKLSCKASGYTFTSYWMKWVKQR |
| | | PGQGLEWIGMIDPSDSETHYNQMFKDKATLTVDKSSSTAYM |
| | | QLSSLTSEDSAVYYCARRWGYDVTMDYWGQGTSVTVSS |
| 10 | Protein | DIVMTQSQKFMSTPVGDRVSVTCKASQNVGTNVAWYQQKP |
| | | GQSPKALIYSASYRYSGVPDRFTGSGSGTDFTLTISNMQSEDL |
| | | ADYFCQQYNTYPLTFGSGTKLEIK |
| 11 | DNA | gaggttcagctgcagcagtctggggcagagcttgtgaagccaggggcctcagtcaagttgtcct |
| | | gcacagcttctggcttcaacattaaagacacctatatacactgggtgaagcagaggcctgaacag |
| | | ggcctggagtggattggaaggattgatcctgcgaatggtaatactaaatatgacccgaagttcca |
| | | gggcaaggccaccataacagcagacacatcctccaacacagcctacctgcagctcagcagcct |
| | | gacatctgaggacactgccgtctgttactgtgctgggtccctctatgattacgacggggccccgttt |
| | | gcttactggggccaagggactctggtcactgtctctgca |
| 12 | DNA | agtattgtgatgacccagactcccaaattcctgcttgtatcagcaggagacagggttaccataacct |
| | | gcaaggccagtcagagtgtgactaatgatgtaacttggtaccaacagaagccagggcagtctcct |
| | | aaactgctgatatactatgcatccaatcgctacactggagtccctgatcgcttcactggcagtggat |
| | | atgggacggatttcactttcaccatcagcactgtgcaggctgaagacctggcagtttatttctgtca |
| | | gcaggattatagctctccgtggacgttcggtggaggcaccaagctggaaatcaaa |
| 13 | DNA | gaggttcagctgcagcagtctggggcagagcttgtgaagccaggggcctcagtcaagttgtcct |
| | | gcacagtttctggcttcaacattaaagacacctatatgcactgggtgaagcagaggcctgaacag |
| | | ggcctggagtggattggaaggattgatcctgcgaatggtaatactaaatatgacccgaagttcca |
| | | gggcaaggccactataacagcagacacatcctccaacacagcctacctgcaactcagcagcct |
| | | gacatctgaggacactgccgtctattactgtgcctcatcctactatgattacgacggttcctggtttg |
| | | cttactggggccaagggactctggtcactgtctctgca |
| 14 | DNA | gaaaatgttctcacccagtctccagcaatcatgtctgcatctccaggggaaaaggtcaccatgacc |
| | | tgcagtgccagctcaagtgtaagttacatgcactggtaccagcagaagtcaagcacctcccccaa |
| | | aatctggatttatgacacatccaaactggcttctggagtcccaggtcgcttcagtggcagtgggtct |
| | | ggaaactcttactctctcacgatcagcagcatggaggctgaagatgttgccacttattactgttttca |
| | | ggggagtgggtacccactcacgttcggctcggggacaaggttggaaataaaa |
| 15 | DNA | gatgtgcagcttcaggagtcgggacctggcctggtgaaaccttctcagtctctgtccctcacctgc |
| | | actgtcactggctactcaatcaccagtgattatgcctggaactggatccggcagtttccaggaaac |
| | | aaactggagtggatgggctacataagctacagtggtagcactagctacaacccatctctcaaaag |
| | | tcgaatctctatcactcgagacacatccaagaaccagttcttcctgcagttgaaatctgtgactactg |
| | | aggacgcagccacatattactgtgcaagaggcgatggttatttctatgctatggactactggggtc |
| | | aaggaacctcagtcaccgtctcctca |
| 16 | DNA | gacattgtgatgacacagtctccatcctccctgagtgtgtcagctggagagaaggtcactatgagc |
| | | tgcaagtccagtcagagtctgttaaacagtggaaatcaaaagagctacttggcctggtaccagca |
| | | gaaaccagggcagcctcctaaactgttgatctacggggcatccactagggaatctggggtccct |
| | | gatcgcttcacaggcagtggatctggaaccgatttcactcttaccatcagcagtgtgcaggctgaa |
| | | gacctggcagtttattcctgtcagaatgatcatagttatcctcccacgttcggtgctgggaccaagc |
| | | tggacctgaaa |
| 17 | DNA | caggtgcaactacaacagtctgggcctcagctggttaggcctggggcttcagtgaagatatcctg |
| | | caaggcttctggttactcattcaccaataactggatgcactgggtgaagcagaggcctggacaag |
| | | gtcttgagtggattggcatgattgatccttccgatagtgaaactaggttaaatcagaaattcaagga |
| | | caaggccacattgactgtagacaaatcctccagcacagcctacatgcaactcagcagcccgaca |
| | | tctgaggactctgcggtctattactgtgcaagggttaactgggaaatctttgactactggggccaag |
| | | gcaccactctcacagtctcctca |
| 18 | DNA | gatattgtgatgactcaggctgcaccctctgtacctgtcactcctggagagtcagtatccatctcctg |
| | | caggtctagtaagagtctcctgaatagaaatggcaacacttacttgtattggttcctgcagaggcca |
| | | ggccagtctcctcagctcctgatatatcggatgtccaaccttgcctcaggagtcccagacaggttc |
| | | agtggcagtgggtcaggaactgctttcacactgagaatcagtagagtggaggctgaggatgtgg |
| | | gtgtttattactgtatgcaacatctagaatatccgctcacgttcggtgctgggaccaagctggagct |
| | | gaaa |
| 19 | DNA | caggtccaactgcagcagcctggggctgagctggtgaggcctggggcttcagtgaagctgtcct |
| | | gcaaggcttctggctacaccttcaccagctactggatgaaatgggtgaagcagaggcctggaca |
| | | aggccttgaatggattggtatgattgatccttcagacagtgaaactcactacaatcaaatgttcaag |
| | | gacaaggccacattgactgtagacaaatcctccagcacagcctacatgcagctcagcagcctga |
| | | catctgaggactctgcggtctattattgtgcaaggaggtggggttacgacgttactatggactactg |
| | | gggtcaaggaacctcagtcaccgtctcctca |
| 20 | DNA | gacattgtgatgacccagtctcaaaaattcatgtccacaccagtaggagacagggtcagcgtcac |
| | | ctgcaaggccagtcagaatgtgggtactaatgtagcctggtatcaacagaaaccagggcaatctc |
| | | ctaaagcactgatttactcggcatcctaccggtacagtggagtccctgatcgcttcacaggcagtg |
| | | gatctgggacagatttcactctcaccatcagcaatatgcagtctgaagacttggcagattatttctgt |
| | | cagcaatataacacctatcctctcacgttcggctcggggacaaagttggaaataaaa |
| 21 | Protein | GFNIKDTY |
| 22 | Protein | IDPANGNT |
| 23 | Protein | AGSLYDYDGAPFAY |
| 24 | Protein | QSVTND |
| 25 | Protein | YAS |
| 26 | Protein | QQDYSSPWT |
| 27 | Protein | ASSYYDYDGSWFAY |
| 28 | Protein | SSVSY |
| 29 | Protein | DTS |
| 30 | Protein | FQGSGYPLT |
| 31 | Protein | GYSITSDYA |
| 32 | Protein | ISYSGST |
| 33 | Protein | ARGDGYFYAMDY |
| 34 | Protein | QSLLNSGNQKSY |
| 35 | Protein | GAS |
| 36 | Protein | QNDHSYPPT |
| 37 | Protein | GYSFTNNW |
| 38 | Protein | IDPSDSET |
| 39 | Protein | ARVNWEIFDY |
| 40 | Protein | KSLLNRNGNTY |
| 41 | Protein | RMS |
| 42 | Protein | MQHLEYPLT |
| 43 | Protein | GYTFTSYW |
| 44 | Protein | IDPSDSET |
| 45 | Protein | ARRWGYDVTMDY |
| 46 | Protein | QNVGTN |
| 47 | Protein | SAS |
| 48 | Protein | QQYNTYPLT |
| 49 | Protein | EVQLQQSGAELVKPGASVKLSCTXSGFNIKDTYXHWVKQRP |
| | | EQGLEWIGRIDPANGNTKYDPKFQGKATITADTSSNTAYLQL |
| | | SSLTSEDTAVXYCAXSXYDYDGXXFAYWGQGTLVTVSA |
| 50 | DNA | caggtccagctgcagcagtctggacctgagctggtgaagcctggggcttcagtgaggatatcct |
| | | gcaaggcttcaggctacaccttcacagcctactatatacactgggtgaagcagaggcctggaca |
| | | gggacttgagtggattggatggatttatcctggaaatgttaatactaagtacaatgagaagttcaag |
| | | ggcaaggccacagtgactgcagacaaatcctccaacacagcctacatgcagctcagcagcctg |
| | | acctctgaggactctgcggtctatttctgtgcaagggactatggtaacctttattactatgctatggac |
| | | tactggggtcaaggaacctcagtcaccgtctcctcag |
| 51 | DNA | aacattgtgctgacccaatctccagcttctttggctgtgtctctagggcagagggccaccatatcct |
| | | gcagagccagtgaaagtgttgatagttatggcaatagttttatgcactggtaccagcagaaaccag |
| | | gacagccacccaaagtcctcatctatcttgcatccaacctagaatctggggtccctgccaggttca |
| | | gtggcagtgggtctaggacagacttcaccctcaccattgatcctgtggaggctgatgatgctgca |
| | | acctattactgtcagcaaaataatgaggatccgtacacgttcggaggggggaccaagctggaaat |
| | | aaaac |
| 52 | Protein | QVQLQQSGPELVKPGASVRISCKASGYTFTAYYIHWVKQRPG |
| | | QGLEWIGWIYPGNVNTKYNEKFKGKATVTADKSSNTAYMQ |
| | | LSSLTSEDSAVYFCARDYGNLYYYAMDYWGQGTSVTVSS |
| 53 | Protein | NIVLTQSPASLAVSLGQRATISCRASESVDSYGNSFMHWYQQ |
| | | KPGQPPKVLIYLASNLESGVPARFSGSGSRTDFTLTIDPVEAD |
| | | DAATYYCQQNNEDPYTFGGGTKLEIK |
| 54 | Protein | GYTFTAYY |
| 55 | Protein | IYPGNVNT |
| 56 | Protein | ARDYGNLYYYAMDY |
| 57 | Protein | ESVDSYGNSF |
| 58 | Protein | LAS |
| 59 | Protein | QQNNEDPYT |

1. A method for detecting intact multimeric and/or intact trimeric severe acute respiratory syndrome coronavirus 2 (SARS-COV-2) spike(S) protein in a sample, the method comprising:
(i) immobilizing a capture protein comprising an antibody variable region onto a solid surface;
(ii) contacting the sample to the capture protein comprising an antibody variable region immobilized on the solid surface, wherein the immobilized capture protein binds to a first receptor binding domain (RBD) on a first S protein monomer unit of the multimeric and/or trimeric SARS-COV-2 S protein to thereby form a protein complex;
(iii) contacting the protein complex with:
a. a detection protein comprising an antibody variable region, wherein the detection protein binds to a second RBD on a second S protein monomer unit of the captured multimeric and/or trimeric SARS-COV-2 S protein and wherein the detection protein comprises a detectable label; or
b. a detection protein comprising an antibody variable region, wherein the detection protein binds to a second RBD on a second S protein monomer unit of the captured multimeric and/or trimeric SARS-COV-2 S protein and contacting the detection protein with an antibody that binds thereto and comprises a detectable label; and
(iv) detecting the label, wherein the presence of the label is indicative of the presence of intact multimeric and/or intact trimeric SARS-COV-2 S protein in the sample.

2. (canceled)

3. The method of claim 1, wherein after step (ii) the solid surface is washed to remove unbound protein.

4. The method of claim 1, wherein after step (iii) the solid surface is washed to remove unbound protein.

5. The method of claim 1, wherein the sample is pre-treated with a zwittergent for 3 hours at 37° C.

6. The method of claim 1, wherein the sample is contacted to the capture protein in a diluent comprising zwittergent.

7. The method of claim 1, wherein the capture protein and/or the detection protein comprise a fragment variable (Fv).

8. The method of claim 7, wherein the capture protein and/or the detection protein are selected from the group consisting of:
(i) a single chain Fv fragment (scFv);
(ii) a dimeric scFv (di-scFv);
(iii) a diabody;
(iv) a triabody;
(v) a tetrabody;
(vi) a fragment antigen binding (Fab);
(vii) a F(ab′)₂;
(viii) a Fv;
(ix) one of (i) to (viii) linked to a constant region of an antibody, a constant fragment (Fc) or a heavy chain constant domain (C_{H}) 2 and/or C_{H}3; and
(x) an antibody.

9. The method of claim 1, wherein the capture protein and/or the detection protein is an antibody comprising:
(i) a heavy chain variable region (V_{H}) comprising:
(a) a complementarity determining region (CDR) 1 comprising a sequence set forth in SEQ ID NO: 21, SEQ ID NO: 31, SEQ ID NO: 37 or SEQ ID NO: 54;
(b) a CDR2 comprising a sequence set forth in SEQ ID NO: 22, SEQ ID NO: 32, SEQ ID NO: 38 or SEQ ID NO: 55; and
(c) a CDR3 comprising a sequence set forth in SEQ ID NO: 23, SEQ ID NO: 27, SEQ ID NO: 33, SEQ ID NO: 39 or SEQ ID NO: 56; and
a light chain variable region (V_{L}) comprising:
(a) a CDR1 comprising a sequence set forth in SEQ ID NO: 24, SEQ ID NO: 28, SEQ ID NO: 34, SEQ ID NO: 40 or SEQ ID NO: 57;
(b) a CDR2 comprising a sequence set forth in SEQ ID NO: 25, SEQ ID NO: 29, SEQ ID NO: 35, SEQ ID NO: 41 or SEQ ID NO: 58; and
(c) a CDR3 comprising a sequence set forth in SEQ ID NO: 26, SEQ ID NO: 30, SEQ ID NO: 36, SEQ ID NO: 42 or SEQ ID NO: 59, or
(ii) a V_{H} comprising a sequence set forth in SEQ ID NO: 1, SEQ ID NO: 3, SEQ ID NO: 5, SEQ ID NO: 7, SEQ ID NO: 49 or SEQ ID NO: 52; and
a V_{L} comprising a sequence set forth in SEQ ID NO: 2, SEQ ID NO: 4, SEQ ID NO: 6, SEQ ID NO: 8 or SEQ ID NO: 53.

10.-12. (canceled)

13. The method of claim 1, wherein the capture protein and the detection protein are the same.

14. The method of claim 13, wherein the capture protein and the detection protein is an antibody comprising a V_{H} comprising a sequence set forth in SEQ ID NO: 1 and a V_{L} comprising a sequence set forth in SEQ ID NO: 2.

15. The method of claim 1, wherein the capture protein and the detection protein are different.

16. The method of claim 15, wherein:
(i) the capture protein is an antibody comprising a V_{H} comprising a sequence set forth in SEQ ID NO: 1 and a V_{L} comprising a sequence set forth in SEQ ID NO: 2; and the detection protein is an antibody comprising a V_{H} comprising a sequence set forth in SEQ ID NO: 5 and a V_{L} comprising a sequence set forth in SEQ ID NO: 6; or
(ii) the capture protein is an antibody comprising a V_{H} comprising a sequence set forth in SEQ ID NO: 1 and a V_{L} comprising a sequence set forth in SEQ ID NO: 2; and the detection protein is an antibody comprising a V_{H} comprising a sequence set forth in SEQ ID NO: 7 and a V_{L} comprising a sequence set forth in SEQ ID NO: 8.

17. The method of claim 1, wherein the detectable label is selected from the group consisting of a radiolabel, an enzyme, a fluorescent label, a luminescent label, a bioluminescent label, a magnetic label, a prosthetic group and a contrast agent.

18. The method of claim 17, wherein the detectable label is horseradish peroxidase (HRP).

19. An anti-SARS-COV-2 antibody, the antibody comprising any one of the following:
(i) a V_{H} comprising:
(a) a CDR1 comprising a sequence set forth in SEQ ID NO: 21,
(b) a CDR2 comprising a sequence set forth in SEQ ID NO: 22, and
(c) a CDR3 comprising a sequence set forth in SEQ ID NO: 23; and
a V_{L} comprising:
(a) a CDR1 comprising a sequence set forth in SEQ ID NO: 24,
(b) a CDR2 comprising a sequence set forth in SEQ ID NO: 25, and
(c) a CDR3 comprising a sequence set forth in SEQ ID NO: 26; or
(ii) a V_{H} comprising:
(a) a CDR1 comprising a sequence set forth in SEQ ID NO: 21,
(b) a CDR2 comprising a sequence set forth in SEQ ID NO: 22, and
(c) a CDR3 comprising a sequence set forth in SEQ ID NO: 27; and
a V_{L} comprising:
(a) a CDR1 comprising a sequence set forth in SEQ ID NO: 28,
(b) a CDR2 comprising a sequence set forth in SEQ ID NO: 29, and
(c) a CDR3 comprising a sequence set forth in SEQ ID NO: 30; or
(iii) a V_{H} comprising:
(a) a CDR1 comprising a sequence set forth in SEQ ID NO: 31,
(b) a CDR2 comprising a sequence set forth in SEQ ID NO: 32, and
(c) a CDR3 comprising a sequence set forth in SEQ ID NO: 33; and
a V_{L} comprising:
(a) a CDR1 comprising a sequence set forth in SEQ ID NO: 34,
(b) a CDR2 comprising a sequence set forth in SEQ ID NO: 35, and
(c) a CDR3 comprising a sequence set forth in SEQ ID NO: 36; or
(iv) a V_{H} comprising:
(a) a CDR1 comprising a sequence set forth in SEQ ID NO: 37,
(b) a CDR2 comprising a sequence set forth in SEQ ID NO: 38, and
(c) a CDR3 comprising a sequence set forth in SEQ ID NO: 39; and
a V_{L} comprising:
(a) a CDR1 comprising a sequence set forth in SEQ ID NO: 40,
(b) a CDR2 comprising a sequence set forth in SEQ ID NO: 41, and
(c) a CDR3 comprising a sequence set forth in SEQ ID NO: 42; or
(v) a V_{H} comprising:
(a) a CDR1 comprising a sequence set forth in SEQ ID NO: 54,
(b) a CDR2 comprising a sequence set forth in SEQ ID NO: 55, and
(c) a CDR3 comprising a sequence set forth in SEQ ID NO: 56; and
a V_{L} comprising:
(a) a CDR1 comprising a sequence set forth in SEQ ID NO: 57,
(b) a CDR2 comprising a sequence set forth in SEQ ID NO: 58, and
(c) a CDR3 comprising a sequence set forth in SEQ ID NO: 59; or
(vi) a V_{H} comprising a sequence set forth in SEQ ID NO: 1 and a V_{L} comprising a sequence set forth in SEQ ID NO: 2; or
(vii) a V_{H} comprising a sequence set forth in SEQ ID NO: 3 and a V_{L} comprising a sequence set forth in SEQ ID NO: 4; or
(viii) a V_{H} comprising a sequence set forth in SEQ ID NO: 5 and a V_{L} comprising a sequence set forth in SEQ ID NO: 6; or
(ix) a V_{H} comprising a sequence set forth in SEQ ID NO: 7 and a V_{L} comprising a sequence set forth in SEQ ID NO: 8; or
(x) a V_{H} comprising a sequence set forth in SEQ ID NO: 52 and a V_{L} comprising a sequence set forth in SEQ ID NO: 53, or
(xi) a V_{H} comprising a sequence expressed from or encoded by a nucleic acid comprising SEQ ID NO: 11 and a V_{L} comprising a sequence expressed from or encoded by a nucleic acid comprising SEQ ID NO: 12; or
(xii) a V_{H} comprising a sequence expressed from or encoded by a nucleic acid comprising SEQ ID NO: 13 and a V_{L} comprising a sequence expressed from or encoded by a nucleic acid comprising SEQ ID NO: 14; or
(xiii) a V_{H} comprising a sequence expressed from or encoded by a nucleic acid comprising SEQ ID NO: 15 and a V_{L} comprising a sequence expressed from or encoded by a nucleic acid comprising SEQ ID NO: 16; or
(xiv) a V_{L} comprising a sequence expressed from or encoded by a nucleic acid comprising SEQ ID NO: 17 and a V_{L} comprising a sequence expressed from or encoded by a nucleic acid comprising SEQ ID NO: 18; or
(xv) a V_{H} comprising a sequence expressed from or encoded by a nucleic acid comprising SEQ ID NO: 50 and a V_{L} comprising a sequence expressed from or encoded by a nucleic acid comprising SEQ ID NO: 51.

20.-25. (canceled)

26. A method for determining the suitability of a protein comprising a RBD of a SARS-COV-2 S protein to induce an immune response against SARS-COV-2, the method comprising:
(i) immobilizing a capture antibody onto a solid surface, wherein the capture antibody comprises:
(a) a V_{H} comprising a sequence set forth in SEQ ID NO: 1 and a V_{L} comprising a sequence set forth in SEQ ID NO: 2;
(b) a V_{H} comprising a sequence set forth in SEQ ID NO: 3 and a V_{L} comprising a sequence set forth in SEQ ID NO: 4;
(c) a V_{H} comprising a sequence set forth in SEQ ID NO: 5 and a V_{L} comprising a sequence set forth in SEQ ID NO: 6;
(d) a V_{H} comprising a sequence set forth in SEQ ID NO: 7 and a V_{L} comprising a sequence set forth in SEQ ID NO: 8; or
(e) a V_{H} comprising a sequence set forth in SEQ ID NO: 52 and a V_{L} comprising a sequence set forth in SEQ ID NO: 53;
(ii) contacting the antibody with the protein comprising the RBD of the SARS-CoV-2 S protein to thereby form a protein complex;
(iii) washing the solid surface to remove unbound protein;
(iv) contacting the protein complex with a labelled detection antibody, wherein the labelled detection antibody comprises:
(a) a V_{H} comprising a sequence set forth in SEQ ID NO: 1 and a V_{L} comprising a sequence set forth in SEQ ID NO: 2;
(b) a V_{H} comprising a sequence set forth in SEQ ID NO: 3 and a V_{L} comprising a sequence set forth in SEQ ID NO: 4;
(c) a V_{H} comprising a sequence set forth in SEQ ID NO: 5 and a V_{L} comprising a sequence set forth in SEQ ID NO: 6;
(d) a V_{H} comprising a sequence set forth in SEQ ID NO: 7 and a V_{L} comprising a sequence set forth in SEQ ID NO: 8; or
(e) a V_{H} comprising a sequence set forth in SEQ ID NO: 52 and a V_{L} comprising a sequence set forth in SEQ ID NO: 53; and
(v) detecting the label, wherein detecting the label indicates that the protein comprising the RBD of the SARS-COV-2 S protein is multimeric and in a conformation sufficient to induce an immune response against SARS-COV-2.


SEQUENCE LISTING (59 sequences)

SEQ ID NO: 1 - protein, 121 aa, synthetic construct
  REGION 1..121: note = Heavy chain VH amino acid sequence of antibody 30B8
EVQLQQSGAELVKPGASVKLSCTASGFNIKDTYIHWVKQRPEQGLEWIGRIDPANGNTKYDPKFQGKATITADTSSNTAYLQLSSLTSEDTAVCYCAGSLYDYDGAPFAYWGQGTLVTVSA

SEQ ID NO: 2 - protein, 107 aa, synthetic construct
  REGION 1..107: note = Light chain VL amino acid sequence of antibody 30B8
SIVMTQTPKFLLVSAGDRVTITCKASQSVTNDVTWYQQKPGQSPKLLIYYASNRYTGVPDRFTGSGYGTDFTFTISTVQAEDLAVYFCQQDYSSPWTFGGGTKLEIK

SEQ ID NO: 3 - protein, 121 aa, synthetic construct
  REGION 1..121: note = Heavy chain VH amino acid sequence of antibody 39E7
EVQLQQSGAELVKPGASVKLSCTVSGFNIKDTYMHWVKQRPEQGLEWIGRIDPANGNTKYDPKFQGKATITADTSSNTAYLQLSSLTSEDTAVYYCASSYYDYDGSWFAYWGQGTLVTVSA

SEQ ID NO: 4 - protein, 106 aa, synthetic construct
  REGION 1..106: note = Light chain VL amino acid sequence of antibody 39E7
ENVLTQSPAIMSASPGEKVTMTCSASSSVSYMHWYQQKSSTSPKIWIYDTSKLASGVPGRFSGSGSGNSYSLTISSMEAEDVATYYCFQGSGYPLTFGSGTRLEIK

SEQ ID NO: 5 - protein, 119 aa, synthetic construct
  REGION 1..119: note = Heavy chain VH amino acid sequence of antibody 6G6
DVQLQESGPGLVKPSQSLSLTCTVTGYSITSDYAWNWIRQFPGNKLEWMGYISYSGSTSYNPSLKSRISITRDTSKNQFFLQLKSVTTEDAATYYCARGDGYFYAMDYWGQGTSVTVSS

SEQ ID NO: 6 - protein, 113 aa, synthetic construct
  REGION 1..113: note = Light chain VL amino acid sequence of antibody 6G6
DIVMTQSPSSLSVSAGEKVTMSCKSSQSLLNSGNQKSYLAWYQQKPGQPPKLLIYGASTRESGVPDRFTGSGSGTDFTLTISSVQAEDLAVYSCQNDHSYPPTFGAGTKLDLK

SEQ ID NO: 7 - protein, 117 aa, synthetic construct
  REGION 1..117: note = Heavy chain VH amino acid sequence of antibody 33E10
QVQLQQSGPQLVRPGASVKISCKASGYSFTNNWMHWVKQRPGQGLEWIGMIDPSDSETRLNQKFKDKATLTVDKSSSTAYMQLSSPTSEDSAVYYCARVNWEIFDYWGQGTTLTVSS

SEQ ID NO: 8 - protein, 112 aa, synthetic construct
  REGION 1..112: note = Light chain VL amino acid sequence of antibody 33E10
DIVMTQAAPSVPVTPGESVSISCRSSKSLLNRNGNTYLYWFLQRPGQSPQLLIYRMSNLASGVPDRFSGSGSGTAFTLRISRVEAEDVGVYYCMQHLEYPLTFGAGTKLELK

SEQ ID NO: 9 - protein, 119 aa, synthetic construct
  REGION 1..119: note = Heavy chain VH amino acid sequence of antibody 31G11
QVQLQQPGAELVRPGASVKLSCKASGYTFTSYWMKWVKQRPGQGLEWIGMIDPSDSETHYNQMFKDKATLTVDKSSSTAYMQLSSLTSEDSAVYYCARRWGYDVTMDYWGQGTSVTVSS

SEQ ID NO: 10 - protein, 107 aa, synthetic construct
  REGION 1..107: note = Light chain VL amino acid sequence of antibody 31G11
DIVMTQSQKFMSTPVGDRVSVTCKASQNVGTNVAWYQQKPGQSPKALIYSASYRYSGVPDRFTGSGSGTDFTLTISNMQSEDLADYFCQQYNTYPLTFGSGTKLEIK

SEQ ID NO: 11 - DNA, 363 bp, synthetic construct
  misc_feature 1..363: note = Heavy chain VH nucleotide sequence of antibody 30B8
  source 1..363: mol_type = other DNA
gaggttcagctgcagcagtctggggcagagcttgtgaagccaggggcctcagtcaagttgtcctgcacagcttctggcttcaacattaaagacacctatatacactgggtgaagcagaggcctgaacagggcctggagtggattggaaggattgatcctgcgaatggtaatactaaatatgacccgaagttccagggcaaggccaccataacagcagacacatcctccaacacagcctacctgcagctcagcagcctgacatctgaggacactgccgtctgttactgtgctgggtccctctatgattacgacggggccccgtttgcttactggggccaagggactctggtcactgtctctgca

SEQ ID NO: 12 - DNA, 321 bp, synthetic construct
  misc_feature 1..321: note = Light chain VL nucleotide sequence of antibody 30B8
  source 1..321: mol_type = other DNA
agtattgtgatgacccagactcccaaattcctgcttgtatcagcaggagacagggttaccataacctgcaaggccagtcagagtgtgactaatgatgtaacttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctccgtggacgttcggtggaggcaccaagctggaaatcaaa

SEQ ID NO: 13 - DNA, 363 bp, synthetic construct
  misc_feature 1..363: note = Heavy chain VH nucleotide sequence of antibody 39E7
  source 1..363: mol_type = other DNA
gaggttcagctgcagcagtctggggcagagcttgtgaagccaggggcctcagtcaagttgtcctgcacagtttctggcttcaacattaaagacacctatatgcactgggtgaagcagaggcctgaacagggcctggagtggattggaaggattgatcctgcgaatggtaatactaaatatgacccgaagttccagggcaaggccactataacagcagacacatcctccaacacagcctacctgcaactcagcagcctgacatctgaggacactgccgtctattactgtgcctcatcctactatgattacgacggttcctggtttgcttactggggccaagggactctggtcactgtctctgca

SEQ ID NO: 14 - DNA, 318 bp, synthetic construct
  misc_feature 1..318: note = Light chain VL nucleotide sequence of antibody 39E7
  source 1..318: mol_type = other DNA
gaaaatgttctcacccagtctccagcaatcatgtctgcatctccaggggaaaaggtcaccatgacctgcagtgccagctcaagtgtaagttacatgcactggtaccagcagaagtcaagcacctcccccaaaatctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaggttggaaataaaa

SEQ ID NO: 15 - DNA, 357 bp, synthetic construct
  misc_feature 1..357: note = Heavy chain VH nucleotide sequence of antibody 6G6
  source 1..357: mol_type = other DNA
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagctacagtggtagcactagctacaacccatctctcaaaagtcgaatctctatcactcgagacacatccaagaaccagttcttcctgcagttgaaatctgtgactactgaggacgcagccacatattactgtgcaagaggcgatggttatttctatgctatggactactggggtcaaggaacctcagtcaccgtctcctca

SEQ ID NO: 16 - DNA, 339 bp, synthetic construct
  misc_feature 1..339: note = Light chain VL nucleotide sequence of antibody 6G6
  source 1..339: mol_type = other DNA
gacattgtgatgacacagtctccatcctccctgagtgtgtcagctggagagaaggtcactatgagctgcaagtccagtcagagtctgttaaacagtggaaatcaaaagagctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattcctgtcagaatgatcatagttatcctcccacgttcggtgctgggaccaagctggacctgaaa

SEQ ID NO: 17 - DNA, 351 bp, synthetic construct
  misc_feature 1..351: note = Heavy chain VH nucleotide sequence of antibody 33E10
  source 1..351: mol_type = other DNA
caggtgcaactacaacagtctgggcctcagctggttaggcctggggcttcagtgaagatatcctgcaaggcttctggttactcattcaccaataactggatgcactgggtgaagcagaggcctggacaaggtcttgagtggattggcatgattgatccttccgatagtgaaactaggttaaatcagaaattcaaggacaaggccacattgactgtagacaaatcctccagcacagcctacatgcaactcagcagcccgacatctgaggactctgcggtctattactgtgcaagggttaactgggaaatctttgactactggggccaaggcaccactctcacagtctcctca

SEQ ID NO: 18 - DNA, 336 bp, synthetic construct
  misc_feature 1..336: note = Light chain VL nucleotide sequence of antibody 33E10
  source 1..336: mol_type = other DNA
gatattgtgatgactcaggctgcaccctctgtacctgtcactcctggagagtcagtatccatctcctgcaggtctagtaagagtctcctgaatagaaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgctcacgttcggtgctgggaccaagctggagctgaaa

SEQ ID NO: 19 - DNA, 357 bp, synthetic construct
  misc_feature 1..357: note = Heavy chain VH nucleotide sequence of antibody 31G11
  source 1..357: mol_type = other DNA
caggtccaactgcagcagcctggggctgagctggtgaggcctggggcttcagtgaagctgtcctgcaaggcttctggctacaccttcaccagctactggatgaaatgggtgaagcagaggcctggacaaggccttgaatggattggtatgattgatccttcagacagtgaaactcactacaatcaaatgttcaaggacaaggccacattgactgtagacaaatcctccagcacagcctacatgcagctcagcagcctgacatctgaggactctgcggtctattattgtgcaaggaggtggggttacgacgttactatggactactggggtcaaggaacctcagtcaccgtctcctca

SEQ ID NO: 20 - DNA, 321 bp, synthetic construct
  misc_feature 1..321: note = Light chain VL nucleotide sequence of antibody 31G11
  source 1..321: mol_type = other DNA
gacattgtgatgacccagtctcaaaaattcatgtccacaccagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatatgcagtctgaagacttggcagattatttctgtcagcaatataacacctatcctctcacgttcggctcggggacaaagttggaaataaaa

SEQ ID NO: 21 - protein, 8 aa, synthetic construct
  REGION 1..8: note = Heavy chain VH CDR1 amino acid sequence of antibodies 30B8 and39E7
GFNIKDTY

SEQ ID NO: 22 - protein, 8 aa, synthetic construct
  REGION 1..8: note = Heavy chain VH CDR2 amino acid sequence of antibodies 30B8 and39E7
IDPANGNT

SEQ ID NO: 23 - protein, 14 aa, synthetic construct
  REGION 1..14: note = Heavy chain VH CDR3 amino acid sequence of antibody 30B8
AGSLYDYDGAPFAY

SEQ ID NO: 24 - protein, 6 aa, synthetic construct
  REGION 1..6: note = Light chain VL CDR1 amino acid sequence of antibody 30B8
QSVTND

SEQ ID NO: 25 - skipped

SEQ ID NO: 26 - protein, 9 aa, synthetic construct
  REGION 1..9: note = Light chain VL CDR3 amino acid sequence of antibody 30B8
QQDYSSPWT

SEQ ID NO: 27 - protein, 14 aa, synthetic construct
  REGION 1..14: note = Heavy chain VH CDR3 amino acid sequence of antibody 39E7
ASSYYDYDGSWFAY

SEQ ID NO: 28 - protein, 5 aa, synthetic construct
  REGION 1..5: note = Light chain VL CDR1 amino acid sequence of antibody 39E7
SSVSY

SEQ ID NO: 29 - skipped

SEQ ID NO: 30 - protein, 9 aa, synthetic construct
  REGION 1..9: note = Light chain VL CDR3 amino acid sequence of antibody 39E7
FQGSGYPLT

SEQ ID NO: 31 - protein, 9 aa, synthetic construct
  REGION 1..9: note = Heavy chain VH CDR1 amino acid sequence of antibody 6G6
GYSITSDYA

SEQ ID NO: 32 - protein, 7 aa, synthetic construct
  REGION 1..7: note = Heavy chain VH CDR2 amino acid sequence of antibody 6G6
ISYSGST

SEQ ID NO: 33 - protein, 12 aa, synthetic construct
  REGION 1..12: note = Heavy chain VH CDR3 amino acid sequence of antibody 6G6
ARGDGYFYAMDY

SEQ ID NO: 34 - protein, 12 aa, synthetic construct
  REGION 1..12: note = Light chain VL CDR1 amino acid sequence of antibody 6G6
QSLLNSGNQKSY

SEQ ID NO: 35 - skipped

SEQ ID NO: 36 - protein, 9 aa, synthetic construct
  REGION 1..9: note = Light chain VL CDR3 amino acid sequence of antibody 6G6
QNDHSYPPT

SEQ ID NO: 37 - protein, 8 aa, synthetic construct
  REGION 1..8: note = Heavy chain VH CDR1 amino acid sequence of antibody 33E10
GYSFTNNW

SEQ ID NO: 38 - protein, 8 aa, synthetic construct
  REGION 1..8: note = Heavy chain VH CDR2 amino acid sequence of antibody 33E10
IDPSDSET

SEQ ID NO: 39 - protein, 10 aa, synthetic construct
  REGION 1..10: note = Heavy chain VH CDR3 amino acid sequence of antibody 33E10
ARVNWEIFDY

SEQ ID NO: 40 - protein, 11 aa, synthetic construct
  REGION 1..11: note = Light chain VL CDR1 amino acid sequence of antibody 33E10
KSLLNRNGNTY

SEQ ID NO: 41 - skipped

SEQ ID NO: 42 - protein, 9 aa, synthetic construct
  REGION 1..9: note = Light chain VL CDR3 amino acid sequence of antibody 33E10
MQHLEYPLT

SEQ ID NO: 43 - protein, 8 aa, synthetic construct
  REGION 1..8: note = Heavy chain VH CDR1 amino acid sequence of antibody 31G11
GYTFTSYW

SEQ ID NO: 44 - protein, 8 aa, synthetic construct
  REGION 1..8: note = Heavy chain VH CDR2 amino acid sequence of antibody 31G11
IDPSDSET

SEQ ID NO: 45 - protein, 12 aa, synthetic construct
  REGION 1..12: note = Heavy chain VH CDR3 amino acid sequence of antibody 31G11
ARRWGYDVTMDY

SEQ ID NO: 46 - protein, 6 aa, synthetic construct
  REGION 1..6: note = Light chain VL CDR1 amino acid sequence of antibody 31G11
QNVGTN

SEQ ID NO: 47 - skipped

SEQ ID NO: 48 - protein, 9 aa, synthetic construct
  REGION 1..9: note = Light chain VL CDR3 amino acid sequence of antibody 31G11
QQYNTYPLT

SEQ ID NO: 49 - protein, 121 aa, synthetic construct
  REGION 1..121: note = Heavy chain VH amino acid consensus sequence of anti-SARS-CoV2antibodies 30B8 and 39E7
  VARIANT 24: note = X is A or V
  VARIANT 34: note = X is M or I
  VARIANT 94: note = X is C or Y
  VARIANT 98: note = X is G or S
  VARIANT 100: note = X is L or Y
  VARIANT 106: note = X is A or S
  VARIANT 107: note = X is P or W
EVQLQQSGAELVKPGASVKLSCTXSGFNIKDTYXHWVKQRPEQGLEWIGRIDPANGNTKYDPKFQGKATITADTSSNTAYLQLSSLTSEDTAVXYCAXSXYDYDGXXFAYWGQGTLVTVSA

SEQ ID NO: 50 - DNA, 364 bp, synthetic construct
  source 1..364: mol_type = other DNA
caggtccagctgcagcagtctggacctgagctggtgaagcctggggcttcagtgaggatatcctgcaaggcttcaggctacaccttcacagcctactatatacactgggtgaagcagaggcctggacagggacttgagtggattggatggatttatcctggaaatgttaatactaagtacaatgagaagttcaagggcaaggccacagtgactgcagacaaatcctccaacacagcctacatgcagctcagcagcctgacctctgaggactctgcggtctatttctgtgcaagggactatggtaacctttattactatgctatggactactggggtcaaggaacctcagtcaccgtctcctcag

SEQ ID NO: 51 - DNA, 334 bp, synthetic construct
  source 1..334: mol_type = other DNA
aacattgtgctgacccaatctccagcttctttggctgtgtctctagggcagagggccaccatatcctgcagagccagtgaaagtgttgatagttatggcaatagttttatgcactggtaccagcagaaaccaggacagccacccaaagtcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtacacgttcggaggggggaccaagctggaaataaaac

SEQ ID NO: 52 - protein, 121 aa, synthetic construct
QVQLQQSGPELVKPGASVRISCKASGYTFTAYYIHWVKQRPGQGLEWIGWIYPGNVNTKYNEKFKGKATVTADKSSNTAYMQLSSLTSEDSAVYFCARDYGNLYYYAMDYWGQGTSVTVSS

SEQ ID NO: 53 - protein, 111 aa, synthetic construct
NIVLTQSPASLAVSLGQRATISCRASESVDSYGNSFMHWYQQKPGQPPKVLIYLASNLESGVPARFSGSGSRTDFTLTIDPVEADDAATYYCQQNNEDPYTFGGGTKLEIK

SEQ ID NO: 54 - protein, 8 aa, synthetic construct
GYTFTAYY

SEQ ID NO: 55 - protein, 8 aa, synthetic construct
IYPGNVNT

SEQ ID NO: 56 - protein, 14 aa, synthetic construct
ARDYGNLYYYAMDY

SEQ ID NO: 57 - protein, 10 aa, synthetic construct
ESVDSYGNSF

SEQ ID NO: 58 - skipped

SEQ ID NO: 59 - protein, 9 aa, synthetic construct
QQNNEDPYT